(19) United States
(12) Patent Application Publication
(10) Pub. No.: US 2024/0138768 A1
(43) Pub. Date: May 2, 2024

(54) NANO SENSOR-EMBEDDED STENT SYSTEM AND METHOD

(71) Applicants: GEORGIA TECH RESEARCH CORPORATION, Atlanta, GA (US); UNIVERSITY OF PITTSBURGH - OF THE COMMONWEALTH SYSTEM OF HIGHER EDUCATION, Pittsburgh, PA (US)

(72) Inventors: Young Jae CHUN, Pittsburgh, PA (US); Moataz Mohsen AH ELSISY, Pittsburgh, PA (US); Woon-Hong YEO, Atlanta, GA (US); Robert HERBERT, Atlanta, GA (US)

(21) Appl. No.: 18/278,712

(22) PCT Filed: Mar. 4, 2022
(86) PCT No.: PCT/US2022/019009

§ 371 Date: Aug. 24, 2023

Related U.S. Application Data
(60) Provisional application No. 63/156,466, filed on Mar. 4, 2021.

(51) Int. Cl. A61B 5/00; A61B 5/02; A61B 5/07; A61F 2/915

(52) U.S. Cl. CPC A61B 5/6862; A61B 5/02007; A61B 5/076; A61B 5/4851; A61F 2/915; A61B 2562/0261; A61B 2562/0285; A61F 2002/91566; A61F 2210/0076; A61F 2240/001

(57) ABSTRACT

An exemplary method and system are disclosed for a fully implantable soft-membrane electronic system that can provide the continuous monitoring of real-time or semi real-time measurements of strain and/or other mechanical properties, via electrical measurements such as capacitance, over an inductive coupling between the measurement system and the implanted device.

### RELATED APPLICATION

This PCT International Application claims priority to, and the benefit of, U.S. Provisional Patent Application No. 63/156,466, filed Mar. 4, 2021, entitled “Nano-Sensor-Embedded Stent System and Method,” which is incorporated by reference herein in its entirety.

### STATEMENT OF GOVERNMENT INTEREST

This invention was made with government support under NIH R03EB028928 awarded by the National Institutes of Health, as well as grant ECCS-2025462 supported by the National Science Foundation (NSF). The government has certain rights in the invention.

### TECHNICAL FIELD

The present disclosure generally relates to non-invasive methods and systems for measuring mechanical properties of tissues within the chambers of the heart or other body organs, e.g., via an electronic implantable device comprising an integrated sensor and implant, e.g., to evaluate the progression of, and/or treat, coronary arterial disease and other cardiovascular diseases.

### BACKGROUND

Atherosclerosis is a common cause of coronary artery disease and a significant factor in broader cardiovascular diseases, which is a leading cause of premature death in the United States. While implantation of a stent is a common treatment of coronary artery disease, a frequent complication is restenosis which can cause the narrowing and stiffening of the stented artery over the stent.

Restenosis is generally defined as a lumen reduction of the artery or vein with a varying degree of severity. Restenosis can progress gradually and often may not exhibit symptoms until one or more of the blockages become severe. Current methods of monitoring for restenosis include periodic imaging or catheterization procedure such as angiography, intravascular ultrasound, optical coherence tomography, and catheter-based measurements.

There is a benefit to improving the detection of restenosis and providing alternative methods to non-invasively measure metrics within the heart or body.

### SUMMARY

An exemplary method and system are disclosed for a fully implantable soft-membrane electronic system that can provide the continuous monitoring of real-time or semi real-time measurements of strain and/or other mechanical properties, via electrical measurements such as capacitance, of the heart or organ of the body that are acquired over an inductive coupling between the measurement system and the implanted device. In the example of an electronic stent, the implanted device includes a mechanical-sensing sensor that is integrated with an implanted device body (e.g., stent body) that, collectively, is configured to expand and contract with a motion of the body organ to provide a mechanical measure of the body organ within which the implanted device is placed. The implanted device body (e.g., stent body) can be configured as an inductive device that serves as an antenna to provide inductive coupling with the measurement system. The inductive device can form, e.g., a resonant circuit, in some embodiments, to which changes in the mechanical measure of the implanted device in the body organ correlate to a change in the electrical measurements of that implanted device.

In some embodiments, the implantable soft-membrane electronic system is employed to measure restenosis.

In some embodiments, the implantable soft-membrane electronic system includes a set of one or more nanomembrane strain sensors that couple to a stent body to form an electronic stent. The implantable soft-membrane electronic system may be configured as a low-profile system that requires minimal invasive implantation to deploy into a blood vessel in a catheterization procedure.

In some embodiments, the implantable soft-membrane electronic system is printable, in whole or in part, from the nanomaterial-based set of soft, membrane strain sensors. The implantable soft-membrane electronic system is configured to utilize a sliding mechanism that can enhance the sensitivity and detection of low strain measurements.

The implantable soft-membrane electronic system may be integrated with a stent body comprising an inductive stent that serves as an antenna or antenna array for wireless sensing operation (e.g., passive wireless sensing operation). The stent body may be fabricated with microscale features to form the antenna or antenna elements.

A study was conducted and evaluated the sensor platform in wireless monitoring restenosis in an artery model and an ex-vivo study in a coronary artery of ovine hearts. The capacitive sensor-based artery implantation system offers unique benefits in wireless, real-time monitoring of stent treatments and arterial health for cardiovascular disease. It was observed that the printed nanomaterials (e.g., aerosol jet printed) could provide improvements for capacitive strain sensing. The wireless electronic stent can also provide unobtrusive monitoring of restenosis in a biomimetic artery model and in an ex-vivo study with ovine hearts. The electronic stent can be configured to operate via inductive coupling operation in a battery-free operation.

In another aspect, a stent is disclosed comprising a mechanical-sensing sensor comprising a plurality of flexible membrane members, including a first membrane member and a second membrane member, wherein the first membrane member is separated from the second membrane member across a dielectric member (e.g., dielectric layer) to form a capacitive structure, wherein the first membrane member is configured to move in a first direction in relation to the second membrane and the second membrane is configured to move in a second direction in relation to the first membrane member different from the first direction to change capacitance defined between the first membrane member and the second membrane member, and wherein the capacitance, or change of capacitance, corresponds to a measure of strain or mechanical properties; and a stent body comprising a plurality of annular struts positioned at intervals in a circumferential direction of the stent body, wherein the mechanical-sensing sensor is coupled, via one or more stretchable interconnects, to the stent body to measure strain of the stent.

In some embodiments, the plurality of annular struts include a first annular strut and a second annular strut, wherein the first annular strut is configured as an electromagnetic radiating body to serve as an antenna for the stent.

In some embodiments, the second annular strut is configured as a second electromagnetic radiating body to serve as an antenna array for the stent with the first annular member.

In some embodiments, the first annular strut is connected to a second annular strut via a plurality of non-conductive interconnects.

In some embodiments, the first annular strut is configured as a wave-shaped strut (e.g., triangular wave, sinusoidal wave, triangular wave offset, sinusoidal wave with offset, asymmetric triangular wave, asymmetric sinusoidal wave, etc.).

In some embodiments, the mechanical-sensing sensor is laminated (e.g., on an inner or outer surface) on the stent body.

In some embodiments, the mechanical-sensing sensor is integrated into the mechanical-sensing sensor.

In some embodiments, each of the plurality of annular struts comprises a laminated structure comprising: a metal core (e.g., strain-less steel); a conductive layer that surrounds the metal core (e.g., Au, Ag, Cu, Al, Zi, Ni, or combination thereof (alloys); and a coating (e.g., an elastomer, e.g., parylene, or an active agent, such as a therapeutic agent).

In some embodiments, the change of capacitance, strain, or mechanical property is employed to measure a state of restenosis of a patient.

In some embodiments, the mechanical-sensing sensor is configured to measure strain or a change in strain.

In some embodiments, the first membrane member has a first protruding structure, wherein the second membrane member has a second protruding structure, and wherein the first protruding structure is parallel to the second protruding structure.

In some embodiments, the first membrane member has a plurality of conductive non-parallel members (e.g., mesh).

In another aspect, a method is disclosed of fabricating a stent of any one of the above embodiments, the method comprising providing a substrate metal core; cutting, via a laser operation, a plurality of bridges in the substrate metal core; filling each of the plurality of bridges with a printed polymer (e.g., polyimide) to form a stretchable interconnect; cutting, the substrate metal core to form the plurality of annular struts positioned at intervals in a circumferential direction of the stent body; electroplating the plurality of annular struts; and coating the plurality of electroplated annular struts.

In some embodiments, the method includes fabricating a first membrane for a strain sensor; fabricating a second membrane for the strain sensor; assembling the first membrane over a first side of a dielectric layer; and assembling the second membrane over a second side of the dielectric layer.

In another aspect, a stent is disclosed comprising a strain sensor comprising a plurality of flexible membrane members, including a first membrane member and a second membrane member, wherein the first membrane member has first protruding structure and the second membrane member has a second protruding structure separated from the first membrane member to form a laminated structure, wherein the first membrane member is configured to move in a first direction in relation to the second membrane and the second membrane is configured to move in a second direction in relation to the first membrane member different from the first direction to change electrical properties defined between the first protruding structure and the second protruding structure, and wherein the electrical properties, or change of electrical properties, corresponds to a measure of strain; and a stent body comprising a plurality of annular struts positioned at intervals in a circumferential direction of the stent body, including a first annular strut and a second annular strut, wherein the strain sensor is coupled, via one or more stretchable interconnects, to the stent body to measure strain of the stent, and wherein the first annular strut is configured as an electromagnetic radiating body to serve as an antenna for the stent.

In some embodiments, the second annular strut is configured as a second electromagnetic radiating body to serve as an antenna array for the stent with the first annular member.

In some embodiments, the stent further includes features of any one of the above embodiments.

In another aspect, a system is disclosed comprising a measurement system comprising an antenna, an acquisition electronics, and a processing unit, wherein the processing unit comprises a processor a memory having instructions stored thereon, wherein execution of the instructions by the processor cause the processor to direct the acquisition electronics to measure and/or interrogate (i) a change in the resonant frequency of an electronic stent implanted in a patient and/or (ii) a change in arterial wall strain properties.

In some embodiments, the electronic stent comprises the stent of any one of above embodiments.

In another aspect, a method is disclosed of monitoring restenosis or progression of restenosis, the method comprising wirelessly interrogating an electronic stent implanted in a subject to determine a first resonant frequency (e.g., during implantation) associated with a strain or mechanical measurement, wherein the electronic stent comprises an inductive and a capacitive component; wirelessly interrogating the electronic stent implanted in the subject to determine a second resonant frequency (e.g., months or years after implantation) associated with the strain or mechanical measurement; determining a change the strain or mechanical measurement as a change between the first resonant frequency and the second resonant frequency to determine a presence of restenosis or the progression of restenosis.

In some embodiments, the wirelessly interrogation is continuously performed.

In some embodiments, the electronic stent includes the stent of any one of the above-discussed embodiments.

### BRIEF DESCRIPTION OF THE DRAWINGS

The skilled person in the art will understand that the drawings described below are for illustration purposes only.

FIG. 1 shows a system comprising an electronic implantable device configured to provide strain and/or other mechanical properties of a body organ (e.g., heart) via a non-invasive inductive electrical measurement in accordance with an illustrative embodiment.

FIG. 2 shows an example implant device body in accordance with an illustrative embodiment

FIGS. 3A-3I show an example configuration of an integrated sensor in accordance with an illustrative embodiment.

FIG. 4 shows another example configuration of the integrated sensor in accordance with an illustrative embodiment.

FIGS. 5A-5C each shows an example method of operation to determine the presence of a disease or condition (e.g., restenosis) using the exemplary system disclosed herein such as the electronic implantable device (e.g., of FIG. 1) in accordance with an illustrative embodiment.

FIG. 6A shows a method of sensor fabrication for an integrated strain sensor configured as a capacitive strain sensor in accordance with an illustrative embodiment.

FIG. 6B shows an example method of fabrication for the implantable device body configured, e.g., as a wireless stent, in accordance with an illustrative embodiment.

FIG. 7 shows an example of a fully implantable, battery-less platform with membrane arterial stiffness sensors for wireless restenosis monitoring employed in a study

FIG. 8 shows a characterization and optimization operation performed on the membrane strain sensors of FIG. 7 in accordance with an illustrative embodiment.

FIG. 9A shows measured strain sensor responses for three evaluated designs in accordance with an illustrative embodiment.

FIG. 9B shows an experimental setup for the cyclic strain testing of the strain sensor of FIG. 7 in accordance with an illustrative embodiment.

FIG. 9C show an experimental setup of the artery model in accordance with an illustrative embodiment.

FIG. 10 shows characterizations of mechanics and functions of strain sensors of FIG. 7 for arterial stiffness monitoring in accordance with an illustrative embodiment.

FIG. 11 shows characterization of restenosis sensing by the strain sensors of FIG. 7 in an artery model of FIG. 9 in accordance with an illustrative embodiment.

FIG. 12 shows experimental results from an Ex-vivo ovine study that evaluated the strain sensors of FIG. 7 in an animal model in accordance with an illustrative embodiment.

FIG. 13A shows an example ovine heart and coronary artery target before implantation (left image) and after implantation (right image) of wireless restenosis sensor.

FIG. 13B shows measurement values from a restenosis sensor implanted in ovine coronary artery.

FIG. 13C shows wireless sensing operation via the implanted sensor placed in the coronary arteries of the ovine heart.

### DETAILED SPECIFICATION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [4] refers to the fourth reference in the list. All references cited and discussed in this specification are incorporated herein by reference in their entireties and to the same extent as if each reference was individually incorporated by reference.

### Example System

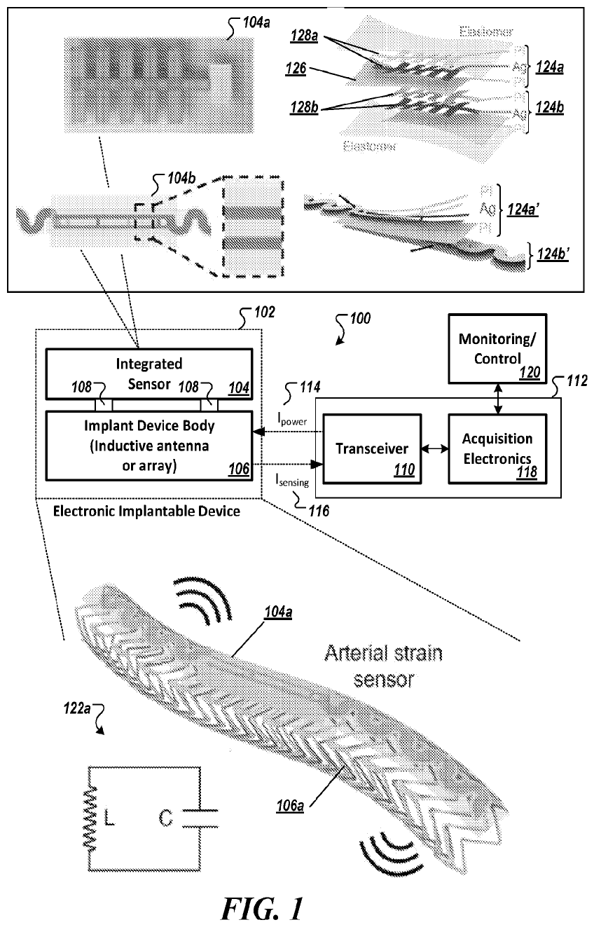

FIG. 1 shows a system 100 comprising an electronic implantable device 102 (shown as an implantable stent device 102a) configured to provide strain and/or other mechanical properties of a body organ (e.g., heart) via a non-invasive inductive electrical measurement in accordance with an illustrative embodiment.

The electronic implantable device 102 includes an integrated sensor 104 (also referred to herein as a “mechanical-sensing sensor”) and an implant device body 106 (e.g., a stent body) that is coupled to the integrated sensor 104 through stretchable or flexible interconnects 108. In the example shown in FIG. 1, the implant device body 106 is configured to wirelessly operate, e.g., via an inductive coupling, with a transceiver 110 of a measurement system 112 that provides inductive interrogation 114 (shown as “I_{power}” 114) to the integrated sensor 104 through the implant device body 106 and receive sensor measurements (shown as “I_{sensing}” 116) from the integrated sensor 104 through the implant device body 106. Stated differently, the implanted device body 106 is configured as an inductive device that serves as an antenna to provide inductive coupling with the measurement system 112.

The inductive device can form, e.g., a resonant circuit 122 (shown in FIG. 1 as an LC circuit 122a) with the integrated sensor to which changes in the mechanical measure of the implanted device 102 via a change an internal motion of components of the integrated sensor, implant device body, or between the sensor the device body, in the body organ correlate to a change in the electrical measurements of that implanted device wherein that electrical measurements or change in the electrical measurements also correlates to a measure of strain or other mechanical properties of the body organ (e.g., blood vessel).

The implantable device body may be configured as a stent such as a balloon-expandable coronary stent, a vascular stent, a ureteral stent, a prostatic stent, a pancreatic or biliary stent, and the like.

The measurement system 112 includes the transceiver 110 and acquisition electronics 118. The acquisition electronics 118 is configured to drive the transceiver 110 and to receive and convert the sensed signal to a data set. The measurement system 112 may operate with a monitoring/control device 120. In some embodiments, the monitoring device 120 includes a data store to store the data set. In other embodiments, the monitoring device 120 is configured to display the acquired data set or to generate an alert.

The monitoring device 120 may be configured as a portable device to provide continuous monitoring of the sensed signal. In other embodiments, the monitoring device 120 may be configured as a medical instrument, e.g., to be employed in a clinician's office, hospital, or medical center, to measure the sensed signal and to direct treatment based on the sensed signal.

In the example shown in FIG. 1, the integrated sensor 104 (shown as 104a) is configured with a plurality of flexible membrane members, including a first membrane member (124a) and a second membrane member (124b) in which the first membrane member (124a) has first protruding structures (128a) and the second membrane member (124b) has second protruding structures (128b) separated from the first membrane member (124a) across a separate or integrated dielectric member 126 (e.g., dielectric layer), e.g., to form a capacitive structure. A second integrated sensor 104 (shown as 104b) is shown, the sensor 104b having flexible membrane members (shown as 124a′ and 124b′).

The first membrane member (e.g., 124a, 124a′) of the integrated sensor (e.g., 104a, 104b) is configured, in some embodiments, to move in a first direction in relation to the second membrane (e.g., 124b, 124b′), and the second membrane (e.g., 124b, 124b′) is configured to move in a second direction in relation to the first membrane member (e.g., 124a, 124a′) different from the first direction to change electrical properties (e.g., capacitance) defined between the first protruding structure and the second protruding structure, e.g., in which the capacitance, or change of capacitance, corresponds to a measure of strain.

### Example Implant Device Body

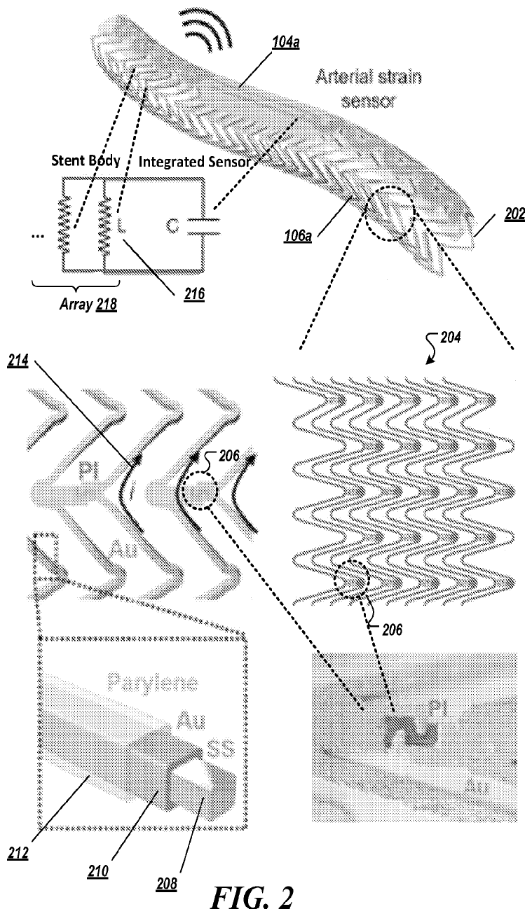

FIG. 2 shows an example implant device body 106 (shown as 106a) in accordance with an illustrative embodiment. In the example of FIG. 2, the implant device body 106a is configured as a stent body that includes a plurality of struts 202 (e.g., annular struts) positioned at periodic intervals to form the device body. At least one of the struts 202a is configured as an electromagnetic radiating body 216 (shown as inductor “L” 216) to serve as an antenna for the device 102/integrated sensor 104, which can be predominately capacitive in a manner or it can be inductive, e.g., to form a resonating circuit. The plurality of struts preferably provide load-bearing functions for the implant device body 106, e.g., load-bearing member in a circumferential direction. Additional struts 202b (see FIG. 2A) can form an antenna array 218 with the first annular member 202a for the device 102/integrated sensor 104.

In the example shown in FIG. 2, the struts 202 are configured as a set of wave-shaped members 204 that are singularly or multiply connected (i.e., via one or multiple connection points) to one another via non-conductive interconnects 206 (previously shown as 108 in FIG. 1). In the example shown in FIG. 2, the non-conductive interconnects 206 is configured as a S-shaped connection comprising of a polymer such as polyimide (also known as Kapton). Other shaped connections may be employed, e.g., serpentine, C shape, U shape, interdigitated structures. The struts 202 may comprise a laminated or layered structure comprising a core (e.g., a metal substrate) (208), a conductive layer 210 that surrounds the core (in whole or in part), and a coating layer 212 that surrounds the conductive layer 210. In the example shown in FIG. 2, the core 208 comprises stainless steel or other medical-grade alloys employed in surgical implants (e.g., Stainless Steel & Inox, Antimagnetic Stainless Steel (such as Dumostar or Dumoxel), titanium, tungsten carbide). The conductive layer 210 provides for the flow of electrical signals and/or electrical energy (shown as 214) for the structure of act in whole an antenna. The conducive layer 210 may be fabricated from gold, silver, platinum, and other suitable medical instrument or surgical grade material that is conductive. The coating layer 212 can be an elastomer, e.g., parylene, or an active agent such as a therapeutic agent.

The implant device body 106a can be laminated, e.g., in a mid-region, or other regions, e.g., via adhesives or via a conductive polymer (e.g., polyimide) with a mechanical-sensing sensor 104a (e.g., a capacitive sensor), e.g., that can measure strain or a change in strain. In some embodiments, the mechanical-sensing sensor 104a can be integrated into the implant device body.

The term “polymer” as used herein refers to a relatively high molecular weight organic compound, natural or synthetic, whose structure can be represented by a repeated small unit, the monomer. Synthetic polymers are typically formed by the addition or condensation polymerization of monomers. The polymers used or produced in the present invention are biodegradable. The polymer is suitable for use in the body of a subject, i.e., is biologically inert and physiologically acceptable, non-toxic, and is biodegradable in the environment of use, i.e., can be resorbed by the body. The term “polymer” encompasses all forms of polymers, including, but not limited to, natural polymers, synthetic polymers, homopolymers, heteropolymers or copolymers, addition polymers, etc.

As discussed herein, a “subject” may be any applicable human, animal, or other organisms, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.” It should be appreciated that, as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to humans (e.g., rat, dog, pig, monkey), etc. It should be appreciated that the subject may be any applicable human patient, for example.

### Example Integrated Sensor

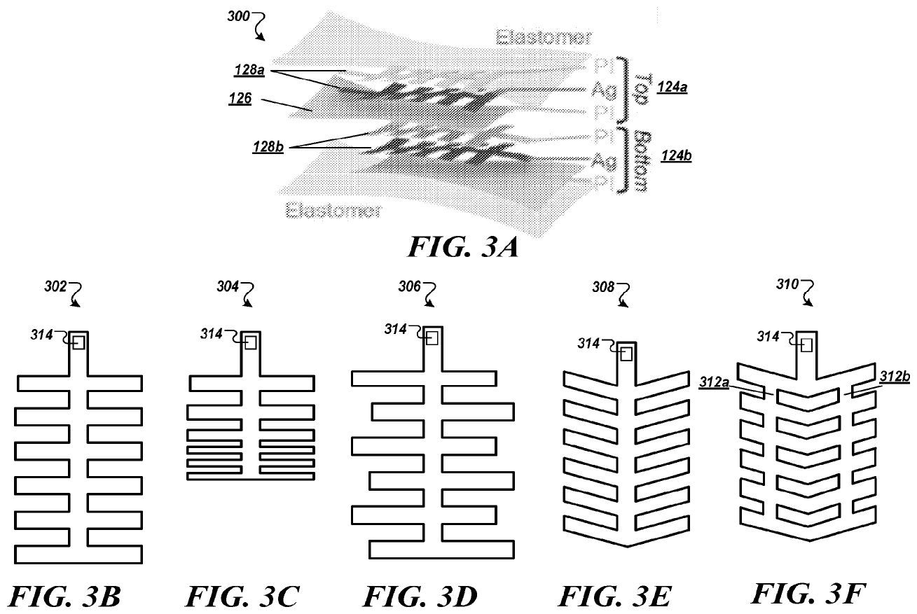

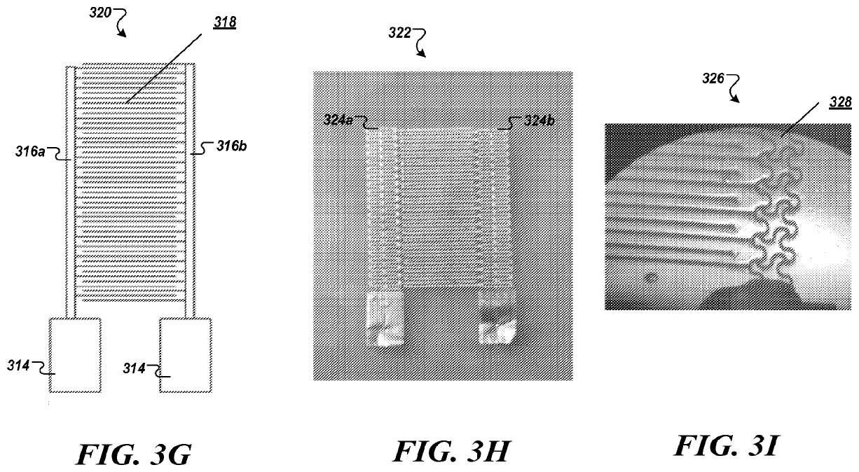

FIGS. 3A-3H each shows an example configuration of an integrated sensor 300 (as an example of an integrated sensor 104a in FIG. 1) in accordance with an illustrative embodiment.

In FIG. 3A, the membrane members (e.g., 124a, 124b, 124a′, 124b′) each has a set of protruding structures (e.g., 128a, 128b), or sheet, that can, e.g., overlap with one another over a dielectric material to form a capacitive structure. The set of protruding structures (e.g., 128a, 128b) may have elements with each having a constant width (e.g., constant finger width) (i.e., the same among each other) (e.g., as shown in 302), or the elements can have non-constant widths that are different among one another. In some embodiments, the protruding structures (e.g., 128a, 128b) may form a patterned sheet.

FIG. 3B-3I each shows an example configuration of a membrane (e.g., plate) of the integrated sensor 300.

FIG. 3B shows a plate 302 having a set of protruding structures (e.g., 128a, 128b) that each has a uniform width and length elements among other nearby structures.

FIG. 3C shows a plate 304 having a set of protruding structures (e.g., 128a, 128b) having varying width and a constant length among other nearby structures.

FIG. 3D shows a plate 306 having a set of protruding structures (e.g., 128a, 128b) having a patterned varying width and/or length elements among nearby structures.

FIG. 3E shows a plate 308 having a set of protruding structures (e.g., 128a, 128b) having angled a patterned varying width and/or length elements among nearby structures.

FIG. 3F shows a plate 310 having a set of protruding structures (e.g., 128a, 128b) having two more branching structures 312 (shown as 312a, 312b) that each has the protruding structures.

FIG. 3G shows a plate 320 having a set of protruding structures (e.g., 128a, 128b) having two more uniform branching structures 316 (shown as 316a, 316b) that has extending elements to form an interdigitated structure.

FIG. 3H shows a plate 322 having a set of protruding structures (e.g., 128a, 128b) having two more non-uniform branching structures 324 (shown as 324a, 324b) that has extending elements to form an interdigitated structure.

FIG. 3H shows a plate 326 having a set of protruding structures (e.g., 128a, 128b) having two more non-uniform meandering branching structures 328 that has extending elements to form an interdigitated structure.

In some embodiments, the two membrane members (e.g., 124a, 124b) are exactly aligned and overlapping with one another to form a capacitive structure. In some embodiments, the two membrane members (e.g., 124a, 124b) can be identical and has a small offset to one another. In yet other embodiments, the two membrane members (e.g., 124a, 124b) can be non-identical to one another.

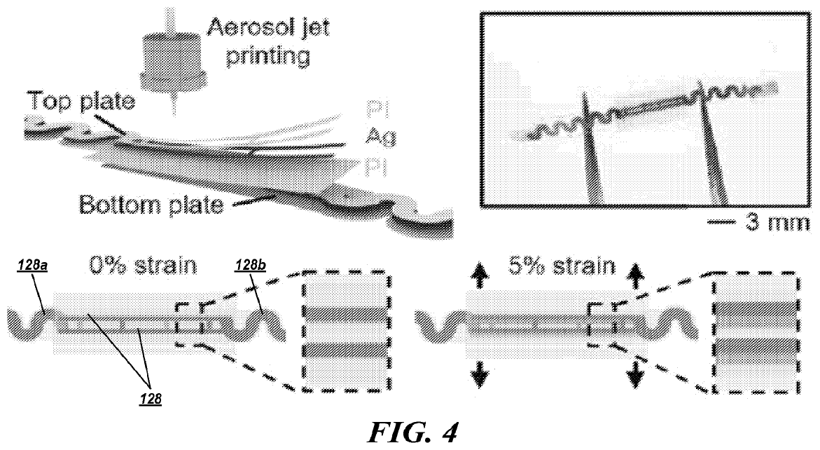

FIG. 4 shows another example configuration of an integrated sensor 400 (as an example of an integrated sensor 104b in FIG. 1) in accordance with an illustrative embodiment.

In FIG. 4, the membrane members (e.g., 124a′, 124b′) each has a set of protruding structures (e.g., 128) that can, e.g., overlap with one another over a dielectric material to form a capacitive structure. In the example shown in FIG. 4, the protruding structures (e.g., 128) form a bridge structure region 402 that has extending serpentine channels (shown as 128a, 128b).

### Example Method of Operation

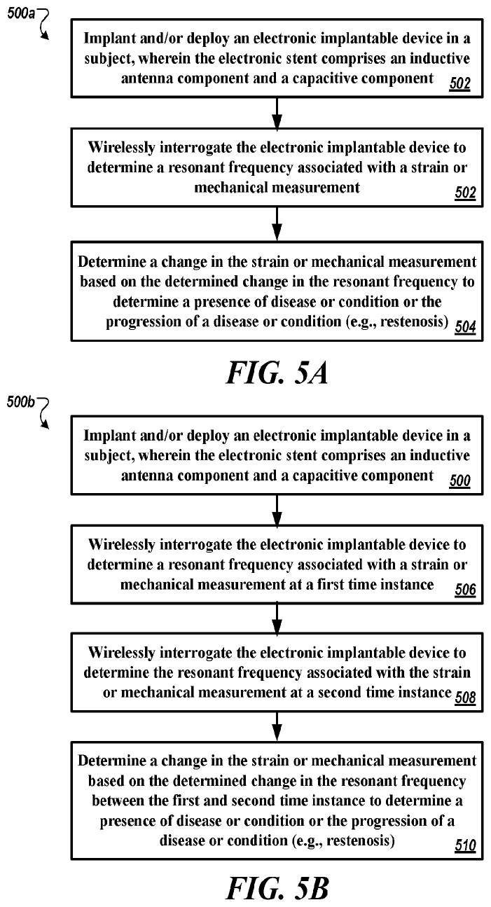

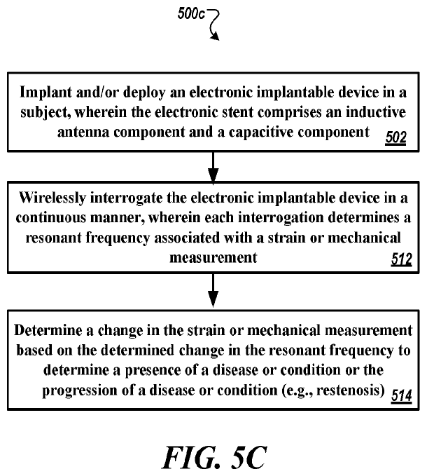

FIGS. 5A-5C each shows an example method 500 (shown as 500a, 500b, and 500c, respectively) of an operation to determine the presence of a disease or condition (e.g., restenosis) using the exemplary system disclosed herein, such as the electronic implantable device (e.g., 102) in accordance with an illustrative embodiment.

Example #1. In the example shown in FIG. 5A, method 500a includes implanting and/or deploying (502) an electronic implantable device (e.g., 102) in a subject in which the electronic implantable device (e.g., 102) comprises an inductive antenna component and a capacitive component. Method 500a includes wirelessly interrogating (502) the electronic implantable device (e.g., 102, 102a, 102b, etc.) to determine a resonant frequency associated with a strain or mechanical measurement. Method 500a includes determining (504) a change in the strain or mechanical measurement based on the determined change in the resonant frequency to determine a presence of disease or condition or the progression of a disease or condition (e.g., coronary arterial disease, atherosclerosis, restenosis, etc.), e.g., to treat said disease or condition.

In some embodiments, the presence of restenosis can be determined as having a low or lower resonance frequency in relation to a reference resonant frequency associated with a healthy or non-restenosis heart.

In some embodiments, the presence of restenosis can be determined as a decrease in resonance frequency, e.g., associated with wall thickening of the vessel walls. In some embodiments, the presence of restenosis can be determined as a decrease in resonance frequency, e.g., associated with increased pressure within a lumen that the electronic implantable device is implanted or deployed.

In some embodiments, the method may assess for an increase in the resonance frequency of the electronic implantable device in the increase in resonance frequency may correspond to a change in strain or mechanical property of a lumen that the electronic implantable device is implanted or deployed, e.g., as a result of a thinning of the lumen or a reduction in pressure in said lumen.

The terms “treat,” “treating,” “treatment,” and grammatical variations thereof, as used herein, include partially or completely delaying, alleviating, mitigating, or reducing the intensity of one or more attendant symptoms of a disorder or condition and/or alleviating, mitigating or impeding one or more causes of a disorder or condition. Treatments according to the invention may be applied preventively, prophylactically, palliatively, or remedially. Prophylactic treatments are administered to a subject prior to onset (e.g., before obvious signs of a lung disorder), during early onset (e.g., upon initial signs and symptoms of a lung disorder), or after an established development of a disease (e.g., a coronary arterial disease, atherosclerosis, restenosis). Prophylactic administration can occur for several days to years prior to the manifestation of symptoms of a disease (e.g., coronary arterial disease, atherosclerosis, restenosis, etc.).

Example #2. In the example shown in FIG. 5B, method 500b includes implanting and/or deploying (502) an electronic implantable device (e.g., 102) in a subject in which the electronic implantable device (e.g., 102) comprises an inductive antenna component and a capacitive component, e.g., as described in relation to FIG. 5A.

The method 500a includes wirelessly interrogating (506) the electronic implantable device (e.g., 102, 102a, 102b, etc.) to determine a resonant frequency associated with a strain or mechanical measurement at a first-time instance. Method 500b includes wirelessly interrogating (508) the electronic implantable device (e.g., 102, 102a, 102b, etc.) to determine a resonant frequency associated with the strain or mechanical measurement at a second-time instance.

Method 500b includes determining (504) a change in the strain or mechanical measurement based on the determined change in the resonant frequency between the first-time instance and the second-time instance to determine a presence of disease or condition or the progression of a disease or condition (e.g., coronary arterial disease, atherosclerosis, restenosis, etc.), e.g., to treat said disease or condition.

In some embodiments, the first-time instance refers to a scan performed during a visit to a clinician to evaluate for restenosis (or coronary arterial disease, atherosclerosis, or other disease or conditions described herein). The second-time instance refers to a scan performed during another visit to a clinician to evaluate for restenosis (or coronary arterial disease, atherosclerosis, or other disease or conditions described herein). The time period between the first and the second time instance may be days, weeks, months, or years apart.

Example #3. In the example shown in FIG. 5C, method 500c includes implanting and/or deploying (502) an electronic implantable device (e.g., 102) in a subject in which the electronic implantable device (e.g., 102) comprises an inductive antenna component and a capacitive component, e.g., as described in relation to FIG. 5A.

Method 500c includes wirelessly interrogating (506) the electronic implantable device (e.g., 102, 102a, 102b, etc.) to determine a resonant frequency associated with a strain or mechanical measurement at a first-time instance. The method 500c includes wirelessly interrogating (508) the electronic implantable device (e.g., 102, 102a, 102b, etc.) in a continuous manner in which each interrogation determines a resonant frequency associated with the strain or mechanical measurement at a second-time instance.

Method 500c includes determining (514) a change in the strain or mechanical measurement based on the determined change in the resonant frequency to determine a presence of disease or condition or the progression of a disease or condition (e.g., coronary arterial disease, atherosclerosis, restenosis, etc.), e.g., to treat said disease or condition.

The method 500c may be performed, e.g., by a remote portable device that may be wearable by the subject or patient, e.g., to monitor for restenosis or other disease or condition as described herein. The remote portable device may be configured to interrogate for a change in resonant frequency responses from the electronic implantable device (e.g., 102, 102a, 102b, etc.) every second, few seconds (e.g., every 10 seconds, 15 seconds, 20 seconds, etc.), minutes (e.g., every minute, 2 minutes, 3 minutes, 4, minutes, 5 minutes, 6 minutes, 7 minutes, 8 minutes, 9 minutes, 10 minutes, 11 minutes, 12 minutes, 13 minutes, 14 minutes, 15 minutes), hours (e.g., every hour or two hours, etc.), or day. Each scan can then be compared to prior scans to determine a decrease in resonant frequency, for example.

Method of Fabrication

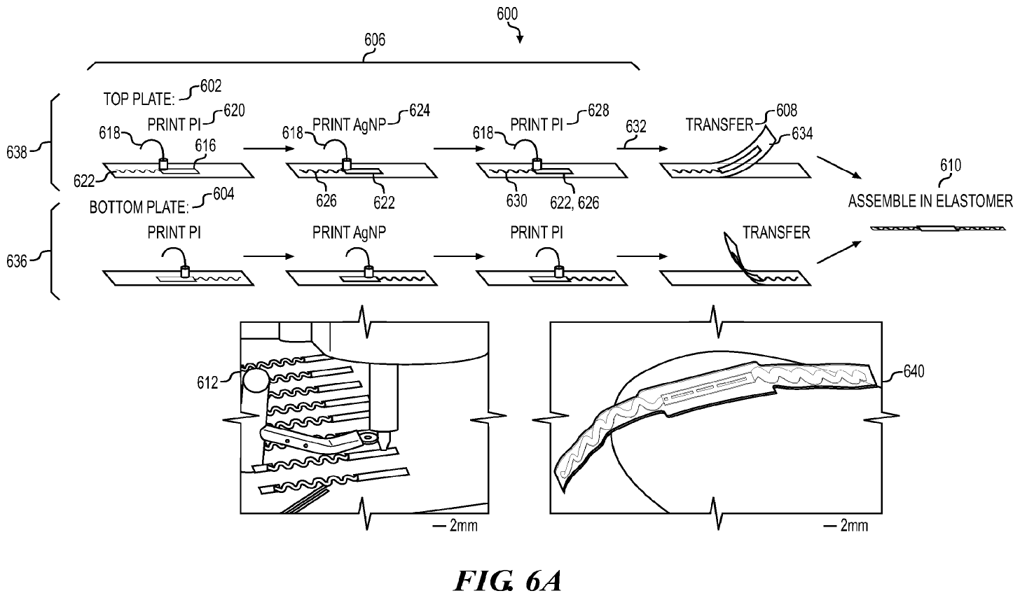

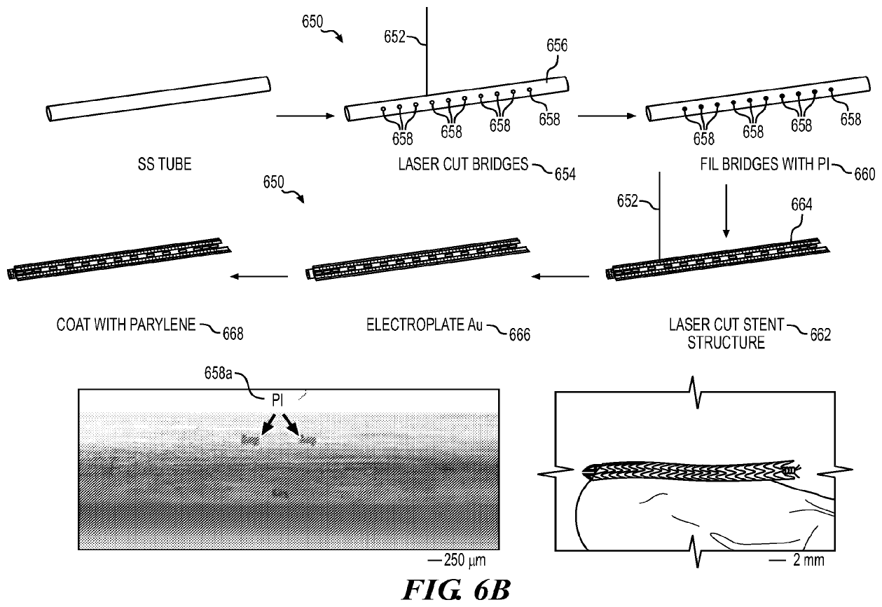

FIG. 6A shows a method 600 of sensor fabrication for an integrated strain sensor 104 configured as a capacitive strain sensor (e.g., 104a or 104b) in accordance with an illustrative embodiment. In the example shown in FIG. 6A, the capacitive strain sensor (104a, 104b) includes a first and second plates (602, 604, respectively) (previously shown as 124a, 124b or 124a′, 124b′). Each of the plates 602, 604 can be printed (606) individually and then be assembled via a transfer and assembly operation (608, 610) to form a capacitive structure that is then assembled and encapsulated in a coating (612). In the example shown in FIG. 6A, the plates 602, 604 can be fabricated via a 3D printing operation, e.g., using an aerosol jet printer. The process 600 may start (not shown) with a substrate 616 (e.g., glass slide) that is coated (not shown) with a layer of polymethyl-methacrylate or other sacrificial later (e.g., photoresist). In some embodiments, a spin-coating operation can be employed to perform the coating, and the coating is then cured.

The aerosol jet printer 618 then prints 620 (shown as “Print PI” 620) a first polyimide layer 622 (e.g., bottom layer) on the coated substrate 616 using a polyimide-ink. The polyimide layer 622 can then be cured and plasma-treated (not shown).

The aerosol jet printer 618 then prints 624 (shown as “Print AgNP” 624) a conductive layer 626, e.g., comprising silver nanoparticles, over the polyimide layer 622. The assembly comprising the printed conductive layer 626 and fabricated layers (e.g., 622) can then be sintered, welded, adhered, or otherwise joined via laser, acoustic, etc.

The aerosol jet printer 618 then prints 628 (shown as “Print PI” 628) a second polyimide layer 630 over the conductive layer 626 shown comprising layers 622, 626. The polyimide layer 630 can then be cured and plasma-treated (not shown).

After printing, the PMMA layer (or sacrificial layer) can be removed or dissolved (632), e.g., via a solution bath. The printed sensor plate 634 can then be transferred 608 (e.g., manually transferred) onto an elastomer layer (e.g., thin elastomer film) and aligned on said layer.

The second plate 604 can be fabricated (636) using similar fabrication processes 638 to fabricate the first plate 602. In some embodiments, the process is performed twice, e.g., where the first and second plates (602, 604) are identical. In other embodiments, the process is performed in parallel and serial for each respective design.

During the assembly operation 610, the first plate 604 can be placed on a thin elastomer layer (e.g., elastomer film, not shown), and then the first plate 602 can be placed over the second plate 604 while aligning the overlapping fingers (e.g., protruding structures 128a, 128b) of the two plates. A second thin elastomer layer (e.g., elastomer film, not shown) can then be joined to, e.g., placed on top of, the sensor, e.g., comprising the top and bottom plates 602, 604) to sandwich the two plates (602, 604) between two elastomer films (not shown) in the coating operation 612. The elastomer layers (e.g., a resin comprising polydimethylsiloxane (PDMS; Sylgard 184, Dow Corning) and poly(styrene-isoprene-styrene) (SIS)) can then be sealed at the edges (e.g., via uncured elastomer or adhesive).

The fabricated sensor 640 can then be connected (e.g., laminated) or integrated into the implant device body 106 (e.g., a stent body 106a). The contacts of the fabricated sensor 640 can be connected to contacts of the implant device body 106 via a conductive paint (e.g., silver paint). The conductive paint may be further coated with a sealing layer (e.g., another elastomer layer). Other conductive means may be employed, e.g., soldering.

FIG. 6B shows an example method 650 of fabrication for the implantable device body 106 configured, e.g., as a wireless stent (e.g., 106a) in accordance with an illustrative embodiment. The implant device body 106a is configured, in an embodiment, as a stent body that includes a plurality of struts 202 (e.g., annular struts) positioned at periodic intervals to form the device body, e.g., as described in relation to FIG. 2. At least one of the struts 202a is configured as an electromagnetic radiating body to serve as an antenna or antenna array for the device. The wireless stent 106a may be fabricated via a set of laser-cutting operations. In some embodiments, water-cutting or acoustic-cutting operations may be used.

In the example shown in FIG. 6B, a femtosecond laser (652) may first laser machine 654 (shown as “Laser cut bridges” 654) a substrate 656 (e.g., steel tube) to remove material at locations (658) correcting to the connectors (e.g., 108, 206). The machined surface can then be polished, e.g., electropolished.

The operation 650 may then include coating 660 (shown as “Fill bridges with PI” 660) polyimide or other connector material onto the tubing, including at least at the locations 658. The coating can then be cured (not shown). The coating process can be repeated multiple times to get the desired connector thickness. The tubing surface can then be sanded and/or polished to remove excess coated material to have only the polyimide or connector material in the precut connector locations 658 (see 658a for an example embodiment of an S-shaped connector).

The operation 650 may then include laser machining 662 (shown as “Laser cut stent structure” 662), e.g., via the femtosecond laser 652 to form the stent structure via a series of patterned cutting operations. The machined surface can then be electropolished to provide the core substrate 664 (e.g., 208 of FIG. 2).

The operation 650 may then include forming 664 (shown as “Electroplate AU” 664) a conductive layer (e.g., 210) over the core substrate 664, e.g., via an electroplating or electrodeposition operation. The layer (e.g., 210) may be 1 and 50 μm thick, e.g., 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm. In some embodiments, the layer (e.g., 210) may be thicker than 50 μm.

The operation 650 may then include forming 668 a coating layer (e.g., 212) over the conductive layer (e.g., 210). In some embodiments, the coating layer (e.g., 212) is formed of an elastomer, e.g., parylene. In some embodiments, additional processing may be performed to treat the coating layer to embed an active agent such as a therapeutic agent in the elastomer. In other embodiments, the active agent is the coating layer. The term “active agent” as used herein can refer to a chemical compound, composition, or organism that has a biological effect. The terms also encompass pharmaceutically acceptable, pharmacologically active derivatives of active agents specifically mentioned herein, including, but not limited to, salts, esters, amides, prodrugs, active metabolites, isomers, fragments, analogs, organisms, and the like. When the term “active agent” is used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The layer (e.g., 212) may be 1 and 100 μm thick, e.g., 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm. In some embodiments, the layer (e.g., 210) may be thicker than 100 μm.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Inhibit,” “inhibiting,” and “inhibition” mean to decrease an activity, response, condition, disease, or another biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

### Experimental Results and Additional Examples

The study was conducted to develop and evaluate a wireless implantable sensor system, previously referred to as an electronic stent device, to monitor the change in arterial stiffness stemming from restenosis, which measures arterial wall strain. The wireless implantable sensor system can be configured as an implantable, battery-less platform configured with one or more arterial stiffness sensors, e.g., for wireless restenosis monitoring.

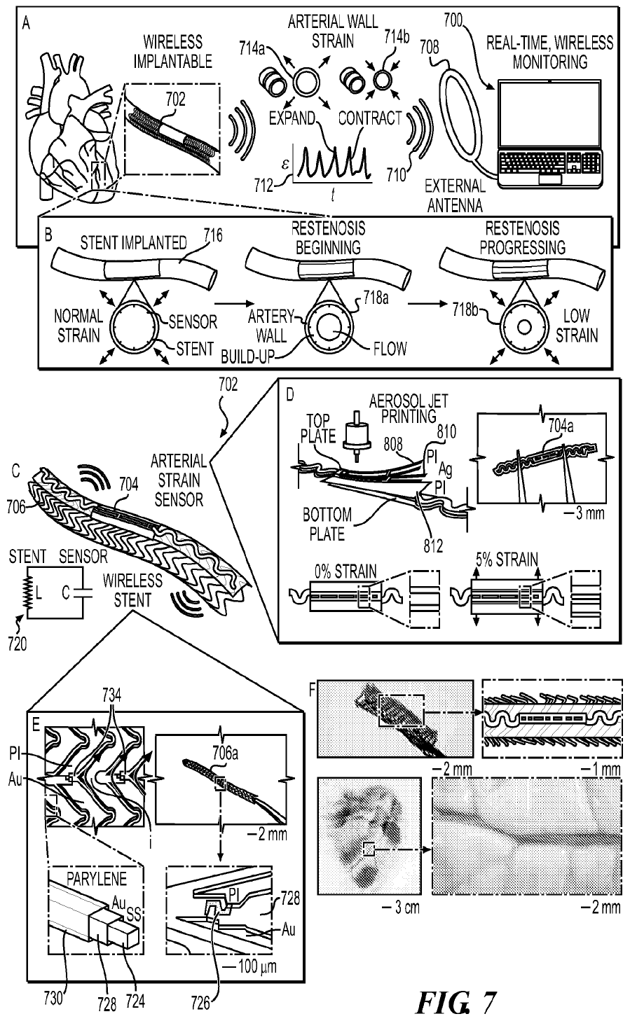

FIG. 7 shows an example of a fully implantable, battery-less platform with membrane arterial stiffness sensors for wireless restenosis monitoring employed in the study

FIG. 7, sub-pane A, illustrates an overview of the monitoring system 700 and an associated method of monitoring arterial strain changes during blood flow. In the study, the implantable sensor 702 (as an example of an electronic implantable device 102) includes a soft strain sensor 704 (as an example of an integrated sensor 104) and stent body 706 (as an example of an implant device body 106) to form an electronic stent. The implantable sensor 702 is configured to be inductively coupled (710) with an external antenna 708 of the monitoring system 700 that can record real-time changes (e.g., plot 712) in arterial wall strain wireless sensed from the implantable sensor 702 as the artery expands and contracts (shown as 714a and 714b, respectively) with the implantable sensor 702 located therein.

The study implanted the stent-based device 702 within an artery 716 via a balloon catheter (not shown). The procedure employed in the study was identical to the current treatment of atherosclerosis of angioplasty and stenting. FIG. 7, sub-pane B, shows the monitoring of arterial stiffness changes with restenosis progression. In FIG. 7, sub-pane B, the gradual build-up within the artery can thicken and stiffen the artery wall (shown as 718a and 718b), resulting in lower strain changes and higher stiffness. The study quantified the change from normal strain to low strain to assess the severity of restenosis in the artery.

FIG. 7, sub-pane C shows an implantable device employed in the study that includes a capacitive strain sensor 704 laminated on an inductive stent 706 to form an LC circuit 720 that is configured to resonant with a resonant frequency that is dependent on strain as asserted on the sensor and stent (704, 706). The soft strain sensor 704 was orientated on the outer surface of the stent body 706, in this example, to detect circumferential strain in the artery. The study operated with an integrated stent and sensor via an inductive coupling mechanism to wirelessly monitor changes in the resonant frequency of the implantable device.

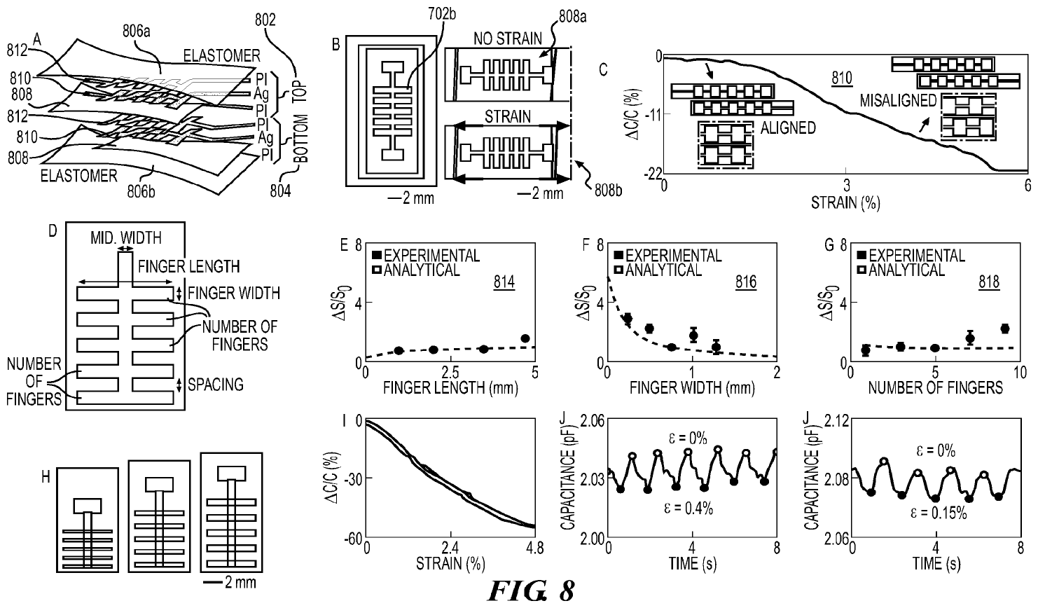

The soft arterial strain sensor was manufactured using aerosol jet printing of silver nanoparticles (AgNP) and polyimide (PI). FIG. 7, sub-pane D shows the separate printing of a top and bottom plate (802, 804) (see FIG. 8) prior to the transfer. The assembled plates are then encapsulated in an elastomer coating to form a parallel-plate capacitor. The study employed the fabrication operation described in FIG. 6A. In FIG. 7, sub-pane D, the overlapping plates slide in opposing directions when the sensor is strained. The sliding of the plates shifts the alignment of overlapping fingers, decreasing the overlapping area and causing a decrease in capacitance. FIG. 7, sub-pane D shows an image of a fabricated miniaturized strain sensor 704 (shown as 704a) that employs stretchable interconnects held by a set of tweezers.

The sensor 704a was integrated onto a wireless stent platform to enable wireless communication from an artery. FIG. 7, sub pane E, shows the wireless stent 706 (shown as 706a) comprising a multi-material structure of stainless steel (SS) (724), polyimide (726), gold (Au) (728), and parylene (730). The stent body 706a was laser machined in the study to have a structure comprising stainless-steel loops (732) and microscale polyimide-connectors (734). The stent body 706 was then electroplated with a layer of gold and then further insulated in parylene. As shown in FIG. 7, sub pane E, the unified structure (comprising 704, 706) facilitates a current flow similar to a solenoid inductor and facilitates the use of the stent body 706 as an inductive antenna. The S-shaped polyimide connectors 734 used in the study can beneficially provide mechanical properties similar to, or enhanced to, conventional stents while providing wireless sensing operation.

FIG. 7, sub pane E, also shows an image of the stent in an assembled form with an enlarged view of the polyimide connectors 726. The integrated set of a stent body (706) and sensor (704) is expandable when in use with a balloon catheter (not shown) that can be employed in a minimally invasive implantation procedure.

FIG. 7, sub pane F, shows the integrated arterial strain sensor and stent device in an expanded configuration. FIG. 7, sub pane G, shows the sensor implanted in the coronary artery in the study. Because of its low-profile form and flexible mechanics, the sensor is readily implantable into narrow arteries.

Characterization and Optimization of Membrane Strain Sensors that Use a Sliding Mechanism

The study evaluated and reconfigured the strain sensor design to provide for high sensitivity and low strain detection that can detect minute changes in arterial stiffness. To facilitate passive sensing, the study evaluated both a capacitive strain sensor and an inductive strain sensor. Numerous studies of soft capacitive strain sensors employ a sensing mechanism based on the Poisson effect, where the dielectric layer changes dimensions under strain [42-52]. However, this mechanism can theoretically limit the sensitivity to around a gauge factor of 1 [53]. To enhance sensitivity and detection of low strains, the exemplary system used in the study was configured to employ a sliding mechanism having overlapping plates located in different membrane layers that can slide relative to each other. The membrane layers (also referred to as plates) included a set of fingers that were fabricated with AgNPs and were aligned to one another to form a soft strain sensor. The exemplary system has been characterized to have high sensitivity, as discussed below.

Example sensors and additional descriptions of said sensor that can be employed are provided in C. M. Boutry, Y. Kaizawa, B. C. Schroeder, A. Chortos, A. Legrand, Z. Wang, J. Chang, P. Fox, Z. Bao, Nature Electronics, 1 (2018) 314-321, which is incorporated by reference herein in its entirety.

FIG. 8 shows a characterization and optimization operation performed on the membrane strain sensors of FIG. 7 in accordance with an illustrative embodiment. Specifically, FIG. 8, sub-pane A, shows an assembly view of the strain sensor comprising a top plate (802) and a bottom plate (804) that are both encapsulated in an elastomeric layer (806a, 806b). Both plates (802, 804) were aerosol jet printed in the study, and each had a bottom polyimide layer (808), a middle AgNP layer (810), and a top polyimide layer (812).

The two identical plates (802, 804) were aligned on top of each other, with the extended bottom PI layer (804) orientated in opposite directions along the length of the sensor (702a). This orientation allowed the two polyimide base layers (802, 804) to be pulled in opposite directions as the elastomer was stretched. FIG. 8, sub-pane B, shows a fabricated strain sensor 702 (shown as 702b) and depicts the sliding mechanism (808a, 808b) where the overlapping fingers of sensor 702b shift during the application of a strain force. As shown in the plot (810) of FIG. 8, sub-pane C, the shifting of plates during the application of a strain was observed in the study to cause a decrease in the capacitance of the sensor 702b as the overlapping areas decreased.

To evaluate and increase the sensitivity of the strain sensor (e.g., 702, 702b), the study investigated the optimization of the strain sensor design (812) with respect to different sensor finger dimensions and configurations. FIG. 8, sub-pane D, shows several sensors that were fabricated with different configurations (shown as 812a, 812b, 812c). The parameters that were adjusted in the study included the finger length (L), the finger width (x), and the number of finger elements (N).

It was observed that the spacing of fingers (y) did not impact the sensitivity but could dictate the maximum strain that can be detected, which may occur when the fingers begin to re-overlap. It was also observed that the middle electrode width (d), as the width of the conductor connecting all the fingers, can be eliminated by offsetting the connection to prevent overlap between two plates. Using these parameters, the study developed an analytical model to predict the capacitance change (ΔC/C) for a given strain per Equation 1.

$$\frac{Δ C}{C} = \frac{d + 2 L N}{N ( x + y ) d + 2 N L x} {L}_{total} ε \qquad ( Eq . 1 )$$

The study evaluated the effects of the finger dimensions using this analytical model. In Equation 1, the model includes the total sensor length (Lₜₒₜₐₗ) and strain (ε). The study identified the finger length, finger width, and the number of fingers as impacting the sensitivity and the size of the sensor. To validate the analytical model, the study printed and assembled strain sensors of different finger dimensions.

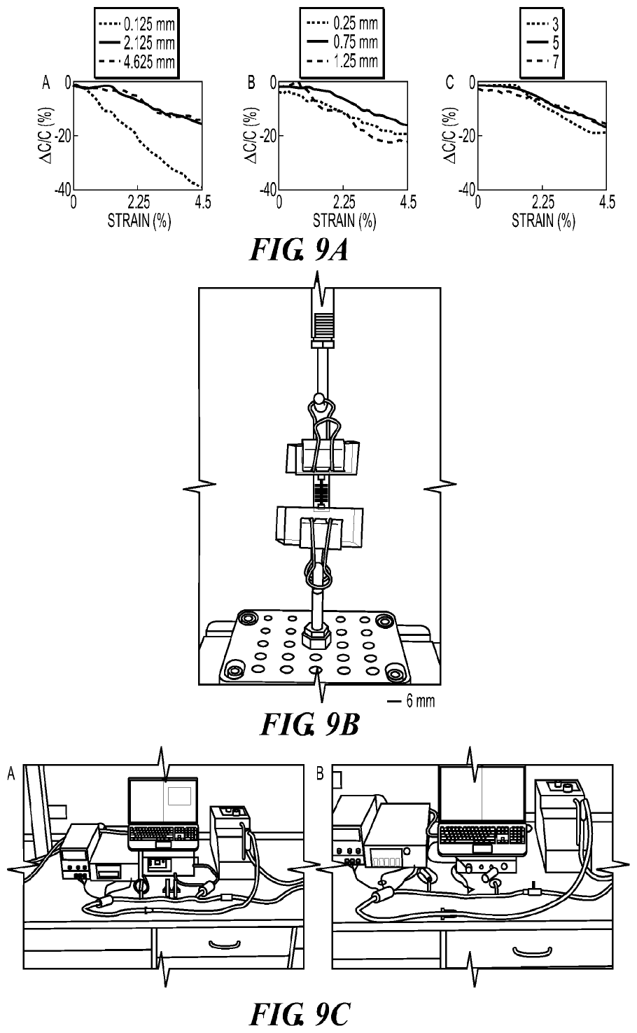

In the study, a baseline sensor (FIG. 8, sub-pane D) was fabricated using five fingers with a width of 0.75 mm and length of 5 mm. All sensors maintained a total length of 25.6 mm, measured from the edges of the polyimide plates. The strain sensors were tested from 0% to 4.8% linear strain, and an overall sensitivity was determined. FIG. 9A shows measured strain sensor responses for three evaluated designs. FIG. 9A, sub-pane A, shows the capacitance change from each sensor with different finger length configurations (0.125, 2.125, and 4.625 mm). FIG. 9A, sub-pane B, shows the change in capacitance for different finger width configurations (0.25, 0.75, and 1.25 mm). FIG. 9A, sub-pane C shows the change in capacitance for difference numbers of fingers (3, 5, and 7 fingers). The baseline sensor displayed a gauge factor of 3.7. FIG. 9B shows an experimental setup for the cyclic strain testing of the strain sensor.

FIG. 8, sub-panes E, F, and G display simulated and experimental capacitance change results of the impact of finger length (814), finger width (816), and the number of fingers (818), respectively. Both analytical and experimental (sub-pane E) results show a trend that the finger length provides a slight increase in sensitivity. The results (sub-pane F) show finger width having a large impact on sensitivity, whereas a smaller finger width provides higher sensitivity. It was observed that a finger width of 0.25 mm could provide a three-times improvement in sensitivity compared to a finger width of 0.75 mm. The results (sub-pane G) show that a larger number of fingers provides a moderate increase in sensitivity. Based on the results of the study, fewer and more narrow fingers may be used to achieve a miniaturized strain sensor while maintaining a high sensitivity.

FIG. 8, sub-pane H, shows three configurations for the implantable sensor 702. Although the narrowest fingers offer the smallest size, it was observed in the study to offer greater sensitivity than the larger counterpart design. In the study, it was observed that the narrowest fingers achieved a sensitivity as high as 10.5 and the sensor capacitance (FIG. 8 sub-pane I). In sub-pane I, it was observed that the sensor achieved a 60% change in capacitance with less than 5% strain. The sensitivity demonstrated by the exemplary system is approximately 10-times greater than the sensitivity typically achieved by soft capacitive strain sensors relying on the Poisson effect design and 3-times greater than a previously reported sliding strain sensor.

Example Microfabrication of Strain Sensor. The study employed an aerosol jet printer (Optomec, Aerosol Jet 200) to print strain sensors. A glass slide was coated with a layer of polymethyl-methacrylate (PMMA; MicroChem) by a spin-coating operation (at 3,000 r.p.m. for 30 s and cured for 3 minutes at 180° C.). The bottom polyimide layer was printed using ink consisting of polyimide (via HD MicroSystems, PI-2545) and 1-methyl-2-pyrrolidinone (NMP, Sigma Aldrich) mixed at a 3.5:1 ratio. The bottom polyimide layer was cured for 1 hour at 240° C. The cured PI was plasma treated prior to the printing via AgNP ink (UTDOTS, AgNP40X). The printed AgNP layer was sintered for 1 hour at 240° C. A top PI layer was printed and cured, similar to the bottom PI layer. The bottom and top plates were printed separately on the same glass slide using identical printing parameters. After printing, the PMMA layer was removed by covering the glass slide and placing it in acetone for over 1 hour. The printed sensor plates were then manually transferred and aligned on elastomer with tweezers. The bottom plate was placed on a thin elastomer film, and the top plate was placed over the top while aligning the overlapping fingers of the two plates. A thin elastomer film was then placed on top of the sensor, sandwiching the two plates between elastomer films. Uncured elastomer was added around the sensor edges to seal the sensor. Both polydimethylsiloxane (PDMS; Sylgard 184, Dow Corning) and poly(styrene-isoprene-styrene) (SIS) were used as elastomer films. PDMS was used to attach the elastomer layers together and to seal the sensor. PDMS was mixed in a 10:1 ratio of base to curing agent. SIS solution was formed based on prior work, where 15 g of poly(styrene-isoprene-styrene) (14% styrene; Sigma-Aldrich) was mixed with 100 ml of propyl acetate (Alfa Aesar). For wired sensing of capacitive sensors, copper (Cu) wire was attached using silver paint. For wireless sensing with a stent, silver paint was used to connect the sensor to the stent, and PDMS was coated over the connections for insulation.

Microfabrication of Wireless Stent. A femtosecond laser (Optec) was used to fabricate the wireless stent from stainless steel tubing (Vita Needle, 304SS 14XX). First, the steel tubing is laser machined to remove material from the connector locations. The machined surfaces were electropolished and rinsed with DI water. PI was then coated onto the tubing and cured at 240° C. for 1 hour. The coating process was repeated twice before sanding the tubing surfaces to remove PI. This allowed PI to only remain in the precut connector locations. Laser machining was performed to cut the remaining stent structure and followed by electropolishing and rinsing. An electroless gold plating solution (Sigma Aldrich) was used to surface plate the stent with approximately a 15 μm thick layer of Au. Electrodeposition was performed with a three-electrode system while the temperature and pH of the solution were maintained at 55° C. and 8. The plated stent was rinsed before insulating the stent with a 30 μm thick layer of parylene.

Table 1 shows a comparison of different capacitive sensor designs [42-52, 54] and the exemplary integrated sensor 702.

TABLE 1

| | Sensitivity | Sensing | |
|---|---|---|---|
| Reference | (AC/C) | Mechanism | Materials |
| This work | Up to 10.4 | Sliding | PI + AgNP |
| [1] | 3.33 | Sliding | PLLA + Mg + PGS |
| [2] | 1.01 | Poisson effect | AgNR + PDMS |
| [3] | 1.45 | Poisson effect | CB + PDMS |
| [4] | 0.7 | Poisson effect | AgNW + PDMS |
| [5] | 0.4 | Poisson effect | CNT + PDMS |
| [6] | 0.99 | Poisson effect | CNT + Silicone |
| [7] | 0.97 | Poisson effect | CNT + PDMS |
| [8] | 1 | Poisson effect | AgNW + PDMS |
| [9] | 3.05 | Poisson effect | Au + 3M VHB tape |
| [10] | 0.83 | Poisson effect | Fabric + silicone |
| [11] | 2 | Poisson effect | AgNW + PDMS |
| [12] | 1.06 | Poisson effect | AgNW + BTO + TPU |

It can be observed that in addition to high sensitivity, the strain sensor of the exemplary system facilitates the detection of small strain changes. It should be appreciated that any of the different capacitive sensor designs can be integrated in an alternative embodiment with implantable device body as described herein.

Indeed, in some embodiments, the exemplary electronic implantable device 102 may employ Poisson effect-based sensors.

FIG. 8, sub-pane J, shows the detection of the sensor of a cyclical measurement comprising a strain change of 0.4%. FIG. 8, sub-pane K, shows the same but at a strain change of 0.15%.

Mechanics and Functions Characterization of Strain Sensors for Arterial Stiffness Monitoring

In the study, criteria based on the sensor optimization results were applied to develop a miniaturized strain sensor for integration with a stent and sensing of arterial stiffness.

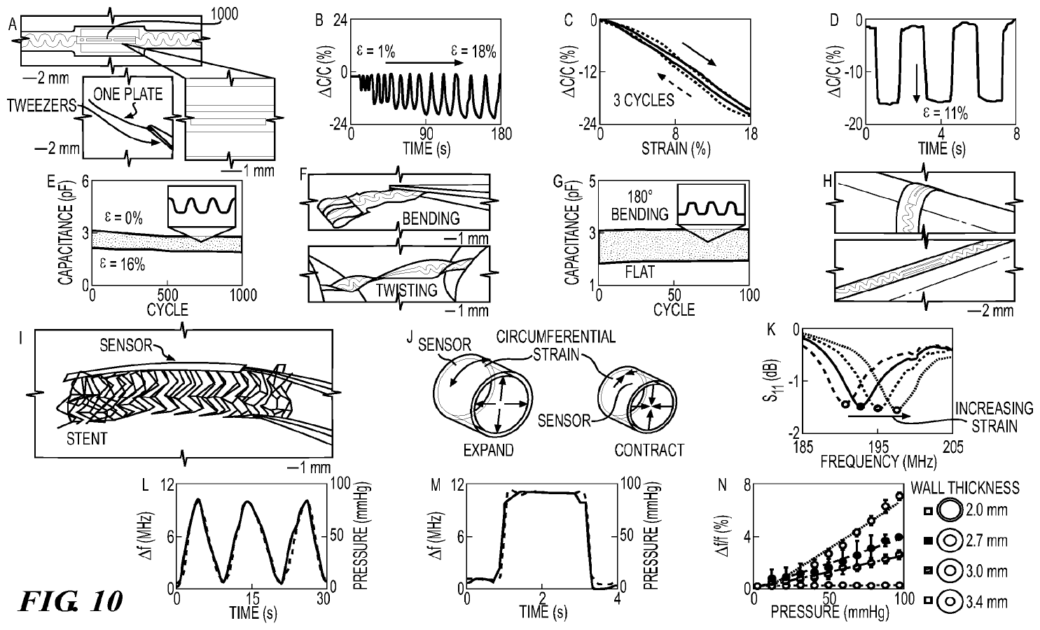

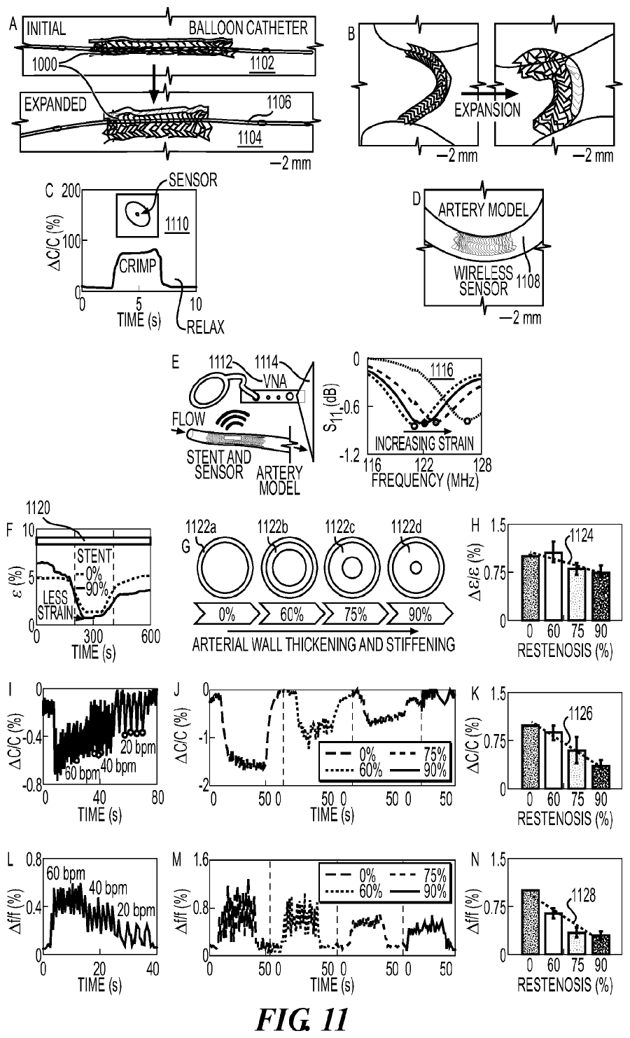

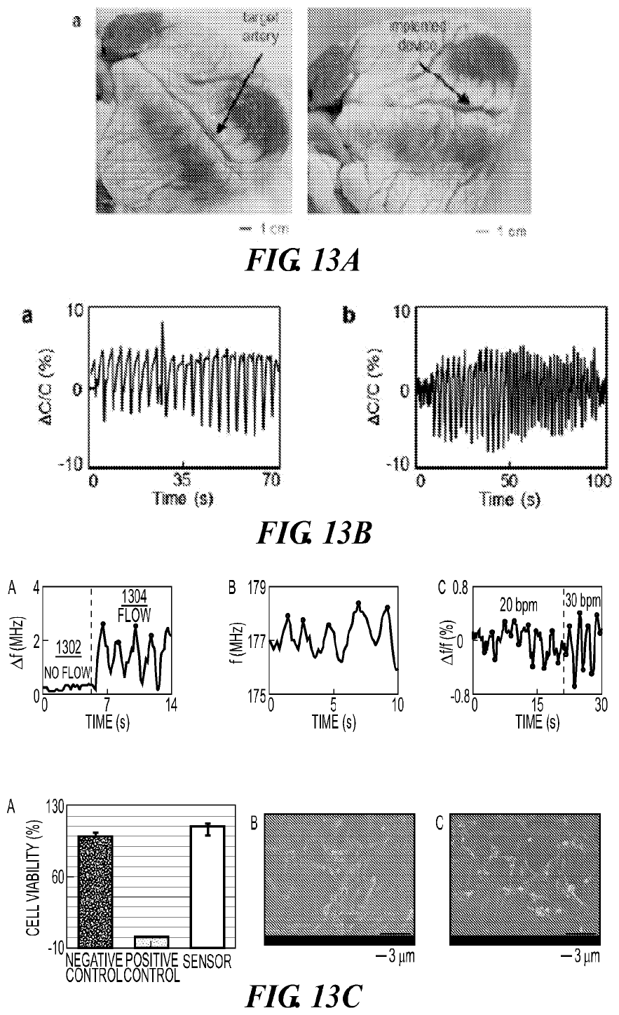

FIG. 10 shows characterizations of mechanics and functions of strain sensors of FIG. 7 for arterial stiffness monitoring in accordance with an illustrative embodiment. FIG. 10, sub-pane A, shows a strain sensor 1000 (as an example of an integrated sensor 104b) for restenosis monitoring, the sensor comprising a thin, printed plate and the overlapping two fingers of the miniaturized sensor. The miniaturized vascular sensor is configured to sense restenosis using stretchable, serpentine interconnections. The sensor fingers can slide vertically to detect strain changes. The sensor 1000 included 2 fingers having a width of 0.3 mm and length of 7.5 mm. FIG. 13, sub-pane A, also displays a side view of a thin printed plate to highlight the low-profile compatibility when implanted. The soft miniaturized strain sensor shows a sensitivity over 1.5 despite a sensing area with a length of 3 mm (FIG. 14).

Characterization of Strain Sensor. Linear strain was applied to the sensors with a motorized test stand (Mark-10 ESM303), while capacitance was measured with an LCR meter (B&K Precision 891). The circumferential strain was applied by embedding sensors in silicone tubing and applying pressure within the tubing. The pressure was measured with a commercial sensor (Honeywell 26PCBFB6G). For strain sensor optimization tests, a linear strain of 4.8% was applied to all sensors. Cyclic stretching and cyclic bending were evaluated using the motorized test stand. Wireless signals from integrated stents and sensors were acquired using a loop antenna connected to a vector network analyzer (VNA; Tektronix TTR506A) to continuously monitor resonant frequency. The resonant frequency was recorded by locating the minimum in the S11 parameter. It was observed that the resistance of the strain sensor remained unchanged during cyclic bending.

Various characteristics of the sensor were evaluated, including response rate, stability, flexibility.

Capacitance Change Dynamics. FIG. 10, sub-pane B, shows a plot of the measured capacitance change of a restenosis sensor 1000 during cyclic strain changes at increasing strain levels. FIG. 10, sub-pane C, shows a plot of the measured capacitance change of the strain sensor 1000 from an applied strain of 0% to 16%. The data indicates a minimal hysteresis characteristic of the sensor due to continuous changes applied to the sensor.

Response time. FIG. 10, sub-pane D, shows the measured change in capacitance by strain sensor 1000 when subjected to a rapid strain change. It can be observed that the sensor displayed immediate changes in capacitance despite sudden stretching to 11% strain.

Stability. FIG. 10, sub-pane E, shows measured capacitance changes of the strain sensor 1000 during cyclic strain from 0% to 16% strain for 1,000 cycles. The sensor was evaluated at strains that more than sufficiently cover the maximum expected artery wall strains of 2-5% [55-57].

Flexibility. FIG. 10, sub-pane F shows the flexibility of thin-film strain sensor 1000 as it is bent, twisted, and folded onto itself without failure. FIG. 10, sub-pane G, shows measured capacitance changes during cyclic 180° bending of strain sensor over a period of time. The sensor shows no change in baseline capacitance after 100 cycles of 180° bending to a radius of 0.5 mm. The data shows the strain sensor 1000 can be configured to have high flexibility. The sensor 1000 can be highly flexible and durable for implantation into an artery and to interface with compliant artery walls.

FIG. 10, sub-panes H and I, show the sensor 100 as a conformal lamination on a curved surface (H) and on a wireless stent (I), respectively. This conformal contact is useful for the integration with a stent to form a low-profile device and to interface unobtrusively with soft artery walls. As a result, the sensor can be well suited to be laminated onto the wireless stent.

FIG. 10, sub-pane J, shows the strain sensor's respective location on the stent device for monitoring circumferential strain from expansion and contraction of a vessel (modeled as a tub). In the evaluation, soft tubing was used to validate the sensing of circumferential, or tubing wall, strain as the tubing expanded and contracted with internal pressure changes. Pressure within the tubing was monitored and controlled, where an increase in pressure caused expansion of the tubing, simulating the distensibility of an expanding artery.

The resonant frequency of the wireless stent and sensor system was measured with a vector network analyzer (VNA) monitoring the S₁₁ parameter. The resonant frequency was identified as the frequency where the S₁₁ parameter is at a minimum.

Wireless signal. FIG. 10, sub-pane K, shows measured wireless S11 signals from the integrated stent and strain sensor. In sub-pane K, it can be observed that there is a shift in resonant frequency from 190 MHz to 198 MHz due to decreasing capacitance of the sensor as the strain is increased.

FIG. 10, sub-pane L, shows the measured resonant frequency of the wireless stent device as it detects circumferential strain from a silicone tube as pressure is changed. It is observed that an increase in pressure (e.g., 90 mm Hg) expands the tubing and causes an increase in strain. Continuously monitoring resonant frequency can thus facilitate the real-time detection of strain changes with a vessel.

FIG. 10, sub-pane M, shows the response time of the wireless sensor in response to an applied change. It can be observed that the sensor 1000 can provide a fast response time.

FIG. 10, sub-pane N, shows a summary of the resonant frequency change as pressure is increased for four different tubing wall thicknesses (2.0 mm, 2.7 mm, 3.0 mm, and 3.4 mm). The soft tubing model could mimic the progression of restenosis where arterial walls thicken, which validated the potential of the wireless sensor for arterial stiffness sensing. It can be observed that the strain sensor provides a decrease in the resonance change with changing pressure as the tubing wall thickness is increased (i.e., stiffens the tubing). That is, a larger change can be observed for thinner tubing walls.

The study also evaluated the effects of the walls. It was observed that a thicker wall tubing could show less strain at a given pressure as compared to a thinner wall tubing.

Restenosis Sensing in Artery Model with Sensors Integrated in a Stent

To implant the sensor (1000) into arteries, the study evaluated the implantation of the integrated stent and sensor (1000) using compatible conventional catheterization procedures. FIG. 9C shows an experimental setup of the artery model (1108). The setup employed a pulsatile pump and pressure monitor to characterize (a) wired sensor capacitance with an LCR meter and (b) wireless sensor resonant frequency with VNA. In the evaluation, the sensor captured the pulsatile waveform of the artery as it expanded and contracted during the changes in flow rate and pressure.

Artery Model. A coronary artery model (1108) was formed by molding silicone (Ecoflex 00-30, Smooth-On). The mold formed an artery with a 3 mm inner diameter, 2 mm thickness, and 100 mm length. Restenosis models included restenosis of 60%, 75%, or 90% at the center of the artery model. To simulate blood viscosity and flow, a 58.5 to 41.5 mixture of water to glycerin was flowed through the artery model (1108) with a pulsatile pump (Harvard Apparatus). A flow rate of 60 mL min⁻¹ at 60 b.p.m. was used to compare sensor signals at different levels of restenosis. The sensor and stent were embedded in the silicone artery model, which is consistent with typical tissue growth over implanted stents. Wired and wireless signals from the sensor were acquired with the LCR meter and VNA, respectively. For restenosis detection, the amplitude of capacitance or resonance changes were compared at each restenosis case and normalized to the amplitude recorded in the normal artery.

Results. FIG. 11 shows characterization of restenosis sensing by the strain sensors of FIG. 7 in an artery model of FIG. 9 in accordance with an illustrative embodiment. To quantify the change in stiffness stemming from restenosis, an optical measurement system was developed to measure arterial strain changes at 0%, 60%, 75%, and 90% restenosis levels. These restenosis levels were selected as 0% for a healthy artery, while restenosis becomes significant as it increases from 60% to 75% and higher. Optical strain measurements were performed to quantify the arterial stiffness along the length of the artery during a flow rate of 60 mL min⁻¹ using 60 b.p.m.

Overall, the artery model validated the use of arterial stiffness sensing and wireless electronics to quantify clinically relevant stages of restenosis. This aspect of the study demonstrated a stent-based arterial stiffness sensor and wireless arterial stiffness sensing [28, 32, 34-36]. By integrating a wireless stent platform with the enhanced soft, capacitive strain sensor, wireless arterial stiffness sensing was achieved while enabling minimally invasive implantation via a catheter. The sensor of the exemplary system can quantify restenosis and offers broader potential for cardiovascular health monitoring.

FIG. 11, sub-pane A, an integrated stent and strain sensor in a contracted state (1102) and an expanded state (1104) with relation to a balloon catheter (1106). It can be observed that the integrated device (1000) was able to be mounted onto a balloon catheter (1106) and expanded. The stent 1100 had a length of 25 mm and started at a 2 mm diameter and expanded up to a 4.8 mm diameter in the study.

FIG. 11, sub-pane B, shows the flexibility aspect of the implantable device (1100) when the device (1100) is in the contracted state (1102) and in the expanded state (1104). In both unexpanded and expanded forms, the device (1100) demonstrated high flexibility that allows it to be guided through narrow, curved arteries to a target location.

FIG. 11, sub-pane C, shows measured capacitance (1110) of the sensor during crimping of stent and sensor onto a balloon catheter. It was observed that the sensor (1000) exhibited mechanical durability and can endure crimping to return to a baseline capacitance after release. Crimping is a common process for stents and ensures proper mounting onto a balloon catheter. To demonstrate arterial stiffness sensing to quantify restenosis, a soft artery model was employed and validated experimentally and computationally [58]. The artery model used a wall thickness of 2 mm and an inner diameter of 4 mm to achieve the biomimetic performance of coronary arteries

FIG. 11, sub-pane D, shows an example device (1000) as expanded, implanted, and deployed in an artery model (1108).

FIG. 11, sub-pane E, shows an example wireless sensing system (1112) configured with a vector network analyzer (VNA) to record the reflection coefficient S11 (1114) of the stent and sensor circuit. The resonant frequency (shown in plot 1116) is observed to change with arterial strain. Pulsatile flow was applied within the artery model, while the resonant frequency of the implanted device was monitored with a VNA and loop antenna (1112). As the flow changed, expansion and contraction of the artery occurred and were detected by changes in resonant frequency, as shown by the frequency sweeps in FIG. 18 sub-pane E.

FIG. 11 sub-pane F shows optically measured strain for a healthy artery and an artery with 90% restenosis. In the study, the stent region (1120) of the artery showed a distinguished decrease in strain since the stent was stiffer than the surrounding artery. The decrease in strain intensified as restenosis progressed. At the stented region, the normal artery showed a strain of 1.5%, while the artery with 90% restenosis showed a strain of 1.1%.

FIG. 11, sub-pane G, shows examples of wall thickening and stiffening as restenosis progresses from 0% to 90% at the four restenosis cases (1122a, 1122b, 1122c, 1122d).

FIG. 11, sub-pane H, shows the average measured strain that was optically measured across the stented region. The data shows a decreasing strain trend (1124) as the restenosis levels increase. Deviations in optically measured strain result from differences in stent stiffness due to fabrication variations. While there is a minor difference between a normal artery and 60% restenosis, there is a decrease in the strain at 75% and 90% restenosis.

FIG. 11, sub-pane I, shows measured sensor capacitance during pulsatile flow within the artery model at three flow rates (60 bpm, 40 bpm, and 20 bpm). The measured change in arterial stiffness was used to validate the wireless arterial stiffness sensor.

FIG. 11, sub-pane J, shows measured sensor capacitance during pulsatile flow at four restenosis levels (1122a, 1122b, 1122c, and 1122d). A smaller change in capacitance occurs as restenosis increases due to stiffening of the artery. The sensor indicated a 0.8% change in capacitance for a flow rate of 60 mL min⁻¹. As restenosis is introduced into the artery model, a decrease in capacitance change was observed from 0% to 90% restenosis for the same flow rate of 60 mL min⁻¹.

FIG. 11, sub-pane K shows a summary of measured capacitance change during the progression of restenosis (1122a, 1122b, 1122c, and 1122d). The capacitance changes were measured as the average amplitude of capacitance change. FIG. 11 sub-pane K shows a decreasing trend (1126) of the capacitive measurement with increasing restenosis.

FIG. 11, sub-pane L, shows measured resonant frequency of wireless sensor 1000 during pulsatile flow in artery model at three different flow rates (e.g., 60 bpm, 40 bpm, 20 bpm).

FIG. 11, sub-pane M, shows measured resonant frequency changes of the sensor at a 60 mL min⁻¹ flow at each restenosis level (1122a, 1122b, 1122c, and 1122d). As restenosis increased, the change in capacitance decreased and caused a decrease in overall resonant frequency change. The inductance of the stent showed minor changes and confirmed that resonance was being shifted by the capacitive sensor.

FIG. 11, sub-pane N, shows a summary of resonant frequency change at each restenosis level. Resonant frequency changes were compared based on the average amplitude change and normalized to the healthy artery case. Similar to wired capacitance measurements, when sensors were misaligned during implantation, the resonant frequency changes can be inverted. FIG. 11 sub-pane N shows a similar decreasing trend (1128) of the wireless measurement (similar to wired measurements) with a large decrease detected from 60% to 75% restenosis. A 35% decrease in resonance changes was observed when introducing a 60% restenosis to a healthy artery. For 75% and 90% restenosis cases, a 63% and 68% decrease in resonance changes were detected.

Table 2. Comparison of implantable sensors for restenosis, occlusion, and arterial stiffness monitoring.

TABLE 2

| | | | | | | Measured | | |
|---|---|---|---|---|---|---|---|---|
| | Target | Wireless/ | Implantation | Distension | | progression of | Real-time | Monitoring |
| Ref | parameter | sensor | method | sensitivity | | occlusion | detection | target |
| This | Arterial | Yes/ | Catheterization | 6.8% | mm⁻¹ | Yes | Wireless - | Restenosis |
| work | stiffness | flow and | | 0.071% | mmHg⁻¹ | | Ex Vivo | |
| | | pressure | | | | | | |
| [36] | Arterial | No | Open | 133% | mm⁻¹ ¹ | No ² | Wired/open | Stiffness |
| | stiffness | | surgery | | | | body - in vivo | |
| [34] | Arterial | No | Open | 0.061% | mmHg⁻¹ ³ | No | Wired/open | Pressure |

| | | | | | | | |
|---|---|---|---|---|---|---|---|
| | stiffness | | surgery | | | body - in vivo | |
| [32] | Blood | Yes/ | Catheterization | No data | No | No | — |
| | pressure | pressure | | | | | |
| | gradient | | | | | | |
| [28] | Blood | Yes/ | Catheterization | No data | No | Wireless/open | Clot ⁵ |
| | pressure | pressure | | | | body - in vivo ⁴ | |
| [35] | Pressure | Yes/ | Open | No data | Yes | Wireless/open | Occlusion |
| | exerted by | pressure | surgery | | | body - in vivo | |
| | artery | | | | | | |

¹ Sensitivity is determined from a scaled sensor signal related to photodetector output voltage.
² Measured stiffness changes from the administration of dopamine.
³ Strain sensor is characterized by comparing capacitance changes with arterial pressure.
⁴ Device implanted in a graft and then implanted using a bypass procedure.
⁵ Measured a signal change from a healthy artery to a fully occluded artery.

An ex-vivo study was conducted with ovine hearts that demonstrated the sensing of stiffness changes in soft arteries.

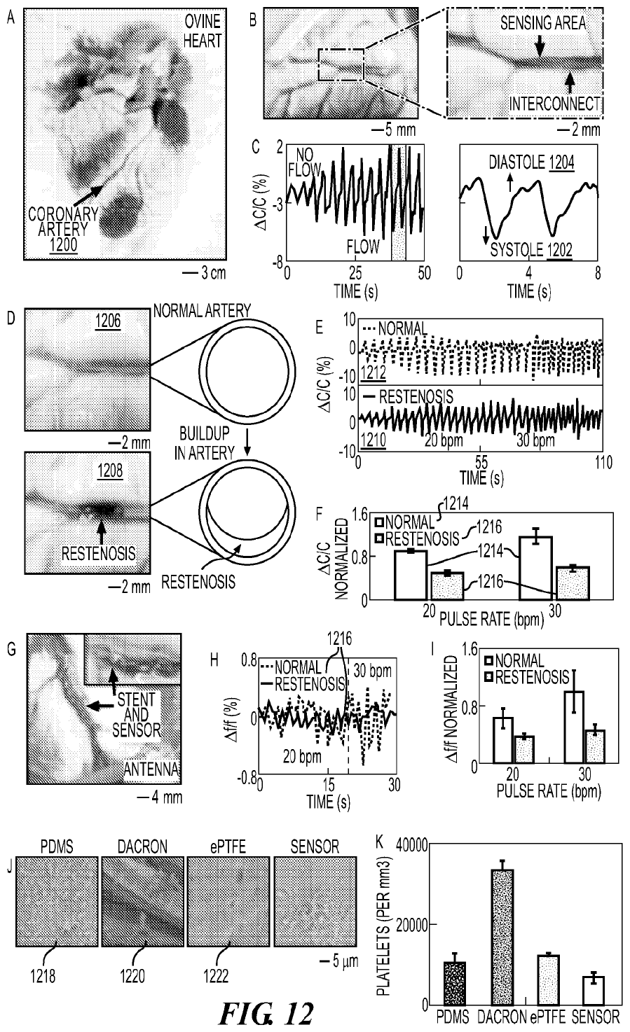

FIG. 12 shows experimental results from an Ex-vivo ovine study that evaluated the strain sensors of FIG. 7 in an animal model in accordance with an illustrative embodiment.

Ex-Vivo Restenosis Monitoring. Ovine hearts were collected for the implantation of stents and sensors into the coronary arteries. A pulsatile pump was connected to the arteries via tubing to enable blood flow. Both a water and glycerin mixture and ovine blood were used for fluid flow in the coronary arteries. Restenosis was simulated by adding silicone (Ecoflex 00-20, Smooth-On) within the artery near the implanted sensor. An LCR meter and VNA were used to measure wired and wireless signals.

Results. FIG. 12, sub-pane A, shows an ovine heart and indicates (via arrow 1200) one of the coronary arteries used for implantation. FIG. 13A shows an example ovine heart and coronary artery target before implantation (left image) and after implantation (right image) of wireless restenosis sensor. The study implanted a wired sensor into a coronary artery to validate sensing of arterial stiffness. FIG. 12, sub-pane B, shows an enlarged view of the soft sensor.

FIG. 12, sub-pane C, shows a plot of example changes in capacitance values as observed between no flow and flow conditions. FIG. 13B shows measurement values from a restenosis sensor implanted in an ovine coronary artery. FIG. 13B (left, sub-pane A) shows the capacitance change of the restenosis sensor during pulsatile flow. FIG. 13B (right, sub-pane B) shows the capacitance change of the restenosis sensor during two flow rates.

In FIG. 12, sub-pane C, the plot displays capacitance values of measurement, including a characteristic waveform of the measured capacitance during the systole and diastole phases 1202, 1204 of the pulsatile wave.

FIG. 12, sub-pane D, shows strain sensors being implanted in animal models with no restenosis 1206 and restenosis 1208 being introduced to the coronary artery of the ovine heart. Silicone was added to the artery to stiffen the region near the implanted sensor to simulate a condition of restenosis.

FIG. 12, sub-pane E, shows measured capacitance values of a sensor (1000) at two flow rates from an artery with and without restenosis. It is observed that a lower change in capacitance occurs in the artery with restenosis due to stiffening of the artery. In both cases (restenosis 1210 and normal 1212), the sensor showed the pulsatile waveform, detection of flow changes, and identified pulse rates similar to the flow setting of 20 and 30 b.p.m. FIG. 12, sub-pane F, shows a plot that summarizes the detected stiffness changes between a healthy artery (1214) and an artery with restenosis (1216) at the two different flow rates. At the higher flow rate, it was observed that a 46% decrease in capacitance change could occur due to restenosis, and at the lower flow rate, it was observed that a 39% decrease in capacitance change could occur due to restenosis.

FIG. 13C shows wireless sensing operation via the implanted sensor placed in the coronary arteries of the ovine heart. FIG. 13C shows a resonant frequency change between no flow condition (2502) (restenosis) and with flow condition (1304) (normal). FIG. 13C, sub-pane B, shows an enlarged view of the resonant frequency changes during flow (1304), showing peak strains. FIG. 13C, sub-pane C shows the resonant frequency changes showing detecting of pulse rate.

FIG. 12, sub-pane G shows the integrated stent and sensor implanted in the artery in the study. FIG. 12, sub-pane H, shows the wirelessly detected resonance changes as measured through an external antenna and VNA monitoring device. It can be observed that a device in an artery with restenosis 1216 exhibited lower changes in resonant frequency compared to the normal artery.

FIG. 12, sub-pane I, shows a plot summarizing the resonance changes. The data aligns well with the trends observed from the wired sensors. Additionally, the soft sensor was evaluated for biocompatibility and hemocompatibility using rat vascular smooth muscle cells and ovine blood. FIG. 13C shows an evaluation of sensor biocompatibility. FIG. 12C, sub-pane A, shows results from an In vitro cell viability tested with rat vascular smooth muscle cell for 24 h by indirect contact methods.

FIG. 12, sub-pane J, displays SEM images of the strain sensor surface along with three established biocompatible materials of polydimethylsiloxane (PDMS) (1218), Dacron (1220), and polytetrafluoroethylene (ePTFE) (1222). Platelet deposition was measured by LDH assay after contact with ovine blood and is summarized in FIG. 12, sub-pane K. The results indicate hemocompatibility of the sensor and the potential of the wireless sensor for arterial stiffness and restenosis monitoring.

Hemocompatibility and Biocompatibility Tests. For the hemocompatibility tests, citrated fresh ovine whole blood was distributed into a vacutainer tube where samples were placed for 2 hours at 37° C. Platelet deposition was quantified by lactate dehydrogenase assay. Biocompatibility was evaluated with rat vascular smooth muscles cells by an indirect contact method for 24 h. Cell viability was analyzed with an MTS assay. Data were normalized to the negative control, which included cells cultured in cell medium only. Cell death was induced in the positive control with 1 M acrylamide dissolved in the cell culture medium.

Discussion

Atherosclerosis, where arteries narrow as artery walls thicken and stiffen, is a leading cause of cardiovascular diseases [1, 2]. In total, cardiovascular diseases are the most common cause of death and account for 31% of deaths [3]. Atherosclerosis can lead to a variety of conditions, including myocardial infarctions, angina, strokes, aneurysms, and gangrene, among others [1]. A common treatment of atherosclerosis is angioplasty and stenting, where the narrowed artery is widened with a balloon catheter and then held open with a stent [4, 5]. Although several million stents are implanted per year, a frequent complication of stenting is restenosis, where the treated artery narrows and stiffens again [6-9]. Restenosis is often defined as a lumen reduction of at least 50% to 70% with varying degrees of severity [10-12]. Restenosis progresses gradually and often causes no symptoms until the blockage becomes severe. While recent advances in drug-eluting stents have reduced restenosis rates to less than 10%, affordability and complication concerns exist for drug-eluting stents [7, 9, 13-15]. Thus, bare metal stents are still often used and have shown restenosis in 17% to 41% of treatments [8, 9]. Notably, restenosis correlates with morbidity and a study has previously indicated restenosis as a predictor of 4 year mortality [12, 16]. Moreover, restenosis may be a risk factor of stent thrombosis, a rarer condition where an acute occlusion occurs of the stented artery [17]. While stent thrombosis occurs in approximately 1% of patients, it shows a mortality rate of up to 45% [18, 19]. Despite the high prevalence of restenosis, monitoring methods are limited to imaging techniques or catheterization, such as angiography, intravascular ultrasound, optical coherence tomography, and catheter-based measurements [12]. Follow-ups occur at varying intervals of time, and, as a result, the progression of restenosis is incompletely monitored. Continuous monitoring would enable early detection and prevention of complications, especially since it has been shown that restenosis rates vary by patient, stent properties, and type of intervention [12, 20].

Continuous, non-invasive monitoring of restenosis enabled by implantable electronics would allow early detection and management of patient health and provide a better understanding of stent designs to minimize restenosis. However, the development of implantable vascular electronics has been restricted due to stringent requirements associated with implantation and operation within soft, narrow arteries. Requirements include a miniaturized, low-profile structure to minimize the impact on blood flow, soft, flexible mechanics to interface with compliant arterial walls, and wireless sensing. Recent advances in soft, wireless electronics offer solutions for the design of implantable vascular sensors [21-29]. Multiple prior works have developed implantable vascular sensors for monitoring blood pressure, and a few have incorporated monitoring of blood flow [26, 28, 30-32]. These works have developed both stent-based sensors and sensors wrapped around the outside of an artery. While some stent-based devices apply a single pressure sensor with a target of monitoring occlusion, it cannot measure the fractional flow reserve (FFR) for restenosis or arterial distensibility [25, 28]. FFR is a widely used measurement to determine the significance of the blockage and is measured by recording pressure both upstream and downstream of restenosis [12, 33]. One work employed two pressure sensors to measure FFR but relies on x-ray imaging to read the sensor [32].

Alternatively to blood pressure monitoring, measuring arterial stiffness or distensibility offers a more direct means to quantify restenosis. As restenosis progresses, artery walls thicken and stiffen, decreasing arterial strain and distensibility. In a similar concept, prior works have investigated sensors to detect the expansion and contraction of arterial walls during blood flow [25, 34-36]. These prior works have developed wireless pressure sensors to be wrapped around the artery to detect arterial occlusion [25, 35]. Additionally, strain sensors, including a capacitive sensor and photonic sensor, have been studied for wrapping around an artery to measure response to blood pressure [34, 36]. While these wrapped sensors demonstrate detection of an occlusion, implantation would be significantly more invasive than conventional catheterization procedures. Moreover, the prior wireless sensors do not demonstrate the ability to quantify restenosis or arterial stiffness and, thus, an implantable device is lacking [25, 35]. Beyond restenosis, an arterial stiffness sensor would offer a broad impact on vascular sensing since arterial stiffness is a significant biomarker of cardiovascular disease and mortality [37-40]. Studies have demonstrated arterial stiffening as a warning sign of cardiovascular morbidity and mortality and is an independent predictor of cardiovascular diseases, including hypertension, atherosclerosis, coronary artery disease, strokes, and heart failures [37-40]. Arterial stiffening is also associated with a heightened risk of organ damage, including to the kidneys and brain, due to the effect on blood flow and pressure [37]. Thus, an arterial stiffness sensor could be readily extended to broader applications, such as continuously monitoring local arterial stiffness to understand the role of arterial stiffness further and as a preventative measure of cardiovascular diseases [41].

In contrast, the instant wireless, soft arterial stiffness sensor electronics may comprise a soft capacitive strain sensor and an electronic stent to monitor arterial stiffness changes stemming from restenosis. The stent-based device is deployable by conventional balloon catheters for minimally invasive implantation and offers unobtrusive sensing of arterial wall strain changes during blood flow. The aerosol jet printed, nanomaterial-based soft strain sensor may employ a sliding mechanism that is investigated and optimized to enhance sensitivity compared to existing soft, capacitive strain sensors. The highly flexible, nanomembrane strain sensor may be integrated onto a multi-material, inductive antenna stent to enable wireless sensing via inductive coupling. The wireless vascular device is demonstrated in a biomimetic coronary artery model to detect restenosis progression. An ex-vivo study with ovine hearts demonstrates restenosis sensing in narrow coronary arteries.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the reference list. For example, Ref. [1] refers to the 1^{st} reference in the list. All references cited and discussed in this specification are incorporated herein by reference in their entireties and to the same extent as if each reference was individually incorporated by reference.

Moreover, the various components may be in communication via wireless and/or hardwire or other desirable and available communication means, systems, and hardware. Moreover, various components and modules may be substituted with other modules or components that provide similar functions.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications as listed below and throughout this document are hereby incorporated by reference in their entirety herein.
[1] P. Libby, J. E. Buring, L. Badimon, G. K. Hansson, J. Deanfield, M. S. Bittencourt, L. Tokgözo\check{g}lu, E. F. Lewis, Nature reviews Disease primers, 5 (2019) 1-18.
[2] N. Torres, M. Guevara-Cruz, L. A. Velàzquez-Villegas, A. R. Tovar, Archives of medical research, 46 (2015) 408-426.
[3] WorldHealthOrganization, (2017).
[4] M. Stuntz, A. Palak, Value in Health, 19 (2016) A641.
[5] A. P. Banning, A. Baumbach, D. Blackman, N. Curzen, S. Devadathan, D. Fraser, P. Ludman, M. Norell, D. Muir, J. Nolan, Heart, 101 (2015) 1-13.
[6] J. A. Ormiston, M. W. Webster, Am Heart Assoc 2016, pp. e004249.
[7] F. Alfonso, R. A. Byrne, F. Rivero, A. Kastrati, Journal of the American College of Cardiology, 63 (2014) 2659-2673.
[8] D. Buccheri, D. Piraino, G. Andolina, B. Cortese, Journal of thoracic disease, 8 (2016) E1150.
[9] S. Mohan, A. Dhall, International journal of angiology, 19 (2010) e66-e72.
[10] L. H. Bonati, J. Gregson, J. Dobson, D. J. McCabe, P. J. Nederkoorn, H. B. van der Worp, G. J. de Borst, T. Richards, T. Cleveland, M. D. Müller, The Lancet Neurology, 17 (2018) 587-596.
[11] B. K. Lal, K. W. Beach, G. S. Roubin, H. L. Lutsep, W. S. Moore, M. B. Malas, D. Chiu, N. R. Gonzales, J. L. Burke, M. Rinaldi, The Lancet Neurology, 11 (2012) 755-763.
[12] D. J. Omeh, E. Shlofmitz, (2019).
[13] G. G. Stefanini, D. R. Holmes Jr, New England Journal of Medicine, 368 (2013) 254-265.
[14] N. Gonzalo, P. W. Serruys, T. Okamura, H. M. van Beusekom, H. M. Garcia-Garcia, G. van Soest, W. van der Giessen, E. Regar, American heart journal, 158 (2009) 284-293.
[15] K. Keikhosravy, A. Zargaran-Yazd, S. Mirabbasi, 2012 Annual International Conference of the IEEE Engineering in Medicine and Biology Society, IEEE2012, pp. 3231-3234.
[16] S. Cassese, R. A. Byrne, S. Schulz, P. Hoppman, J. Kreutzer, A. Feuchtenberger, T. Ibrahim, I Ott, M. Fusaro, H. Schunkert, European heart journal, 36 (2015) 94-99.
[17] M. Joner, A. V. Finn, A. Farb, E. K. Mont, F. D. Kolodgie, E. Ladich, R. Kutys, K. Skorija, H. K. Gold, R. Virmani, Journal of the American College of Cardiology, 48 (2006) 193-202.
[18] R. Reejhsinghani, A. S. Lotfi, Vascular health and risk management, 11 (2015) 93.
[19] D. R. Holmes, D. J. Kereiakes, S. Garg, P. W. Serruys, G. J. Dehmer, S. G. Ellis, D. O. Williams, T. Kimura, D. J. Moliterno, Journal of the American College of Cardiology, 56 (2010) 1357-1365.
[20] A. Kastrati, J. Mehilli, J. Dirschinger, J. Pache, K. Ulm, H. Schühlen, M. Seyfarth, C. Schmitt, R. Blasini, F.-J. Neumann, The American journal of cardiology, 87 (2001) 34-39.
[21] R. Herbert, H. R. Lim, S. Park, J. H. Kim, W. H. Yeo, Advanced Healthcare Materials, (2021) 2100158.
[22] H. R. Lim, H. S. Kim, R. Qazi, Y. T. Kwon, J. W. Jeong, W. H. Yeo, Advanced Materials, 32 (2020) 1901924.
[23] R. Herbert, S. Mishra, H. R. Lim, H. Yoo, W. H. Yeo, Advanced Science, 6 (2019) 1901034.
[24] Y. J. Hong, H. Jeong, K. W. Cho, N. Lu, D. H. Kim, Advanced Functional Materials, 29 (2019) 1808247.
[25] C. M. Boutry, L. Beker, Y. Kaizawa, C. Vassos, H. Tran, A. C. Hinckley, R. Pfattner, S. Niu, J. Li, J. Claverie, Nature biomedical engineering, 3 (2019) 47-57.
[26] M. A. Fonseca, M. G. Allen, J. Kroh, J. White, Tech. Dig. Solid-State Sensor, Actuator, and Microsystems Workshop (Hilton Head 2006), Citeseer 2006, pp. 37-42.
[27] J. Vishnu, G. Manivasagam, Medical Devices & Sensors, 3 (2020) e10116.
[28] X. Chen, B. Assadsangabi, Y. Hsiang, K. Takahata, Advanced Science, 5 (2018) 1700560.
[29] D. Son, J. Lee, D. J. Lee, R. Ghaffari, S. Yun, S. J. Kim, J. E. Lee, H. R. Cho, S. Yoon, S. Yang, ACS nano, 9 (2015) 5937-5946.
[30] J. Park, J.-K. Kim, D.-S. Kim, A. Shanmugasundaram, S. A. Park, S. Kang, S.-H. Kim, M. H. Jeong, D.-W. Lee, Sensors and Actuators B: Chemical, 280 (2019) 201-209.
[31] M. N. Gulari, M. Ghannad-Rezaie, P. Novelli, N. Chronis, T. C. Marentis, 2014 IEEE 27th International Conference on Micro Electro Mechanical Systems (MEMS), IEEE2014, pp. 893-896.
[32] M. N. Gulari, M. Ghannad-Rezaie, P. Novelli, N. Chronis, T. C. Marentis, Journal of Microelectromechanical Systems, 24 (2014) 50-61.
[33] R. Lopez-Palop, E. Pinar, I. Lozano, D. Saura, F. Picó, M. Valdés, European heart journal, 25 (2004) 2040-2047.
[34] P. Bingger, M. Zens, P. Woias, Biomedical microdevices, 14 (2012) 573-581.
[35] S. R. Ruth, M.-g. Kim, H. Oda, Z. Wang, Y. Khan, J. Chang, P. M. Fox, Z. Bao, Iscience, 24 (2021) 103079.
[36] D. Ruh, S. Subramanian, S. Sherman, J. Ruhhammer, M. Theodor, L. Dirk, K. Foerster, C. Heilmann, F. Beyersdorf, H. Zappe, Biomedical optics express, 7 (2016) 3230-3246.
[37] J. A. Chirinos, Journal of cardiovascular translational research, 5 (2012) 243-255.
[38] N. A. Shirwany, M.-h. Zou, Acta Pharmacologica Sinica, 31 (2010) 1267-1276.
[39] Y. S. Oh, Clinical hypertension, 24 (2018) 1-3.
[40] M. Yambe, H. Tomiyama, Y. Hirayama, Z. Gulniza, Y. Takata, Y. Koji, K. Motobe, A. Yamashina, Hypertension Research, 27 (2004) 625-631.
[41] M. Collette, A. Humeau, C. Chevalier, J.-F. Hamel, G. Leftheriotis, Hypertension research, 34 (2011) 578-583.
[42] P. Goel, J. Singh, Journal of Physics D: Applied Physics, 48 (2015) 445303.
[43] V. Tsouti, V. Mitrakos, P. Broutas, S. Chatzandroulis, IEEE Sensors Journal, 16 (2016) 3059-3067.
[44] S. Yao, Y. Zhu, Nanoscale, 6 (2014) 2345-2352.
[45] D. J. Lipomi, M. Vosgueritchian, B. C. Tee, S. L. Hellstrom, J. A. Lee, C. H. Fox, Z. Bao, Nature nanotechnology, 6 (2011) 788-792.
[46] D. J. Cohen, D. Mitra, K. Peterson, M. M. Maharbiz, Nano letters, 12 (2012) 1821-1825.
[47] L. Cai, L. Song, P. Luan, Q. Zhang, N. Zhang, Q. Gao, D. Zhao, X. Zhang, M. Tu, F. Yang, Scientific reports, 3 (2013) 1-9.
[48] F. Xu, Y. Zhu, Advanced materials, 24 (2012) 5117-5122.
[49] R. Nur, N. Matsuhisa, Z. Jiang, M. O. G. Nayeem, T. Yokota, T. Someya, Nano letters, 18 (2018) 5610-5617.
[50] O. Atalay, Materials, 11 (2018) 768.
[51] S.-R. Kim, J.-H. Kim, J.-W. Park, ACS applied materials & interfaces, 9 (2017) 26407-26416.
[52] Y. Fan, Z. Shen, X. Zhou, Z. Dan, L. Zhou, W. Ren, T. Tang, S. Bao, C. Nan, Y. Shen, Advanced Materials Technologies, (2021) 2101190.
[53] J. Shintake, T. Nagai, K. Ogishima, Frontiers in Robotics and AI, 6 (2019) 127.
[54] C. M. Boutry, Y. Kaizawa, B. C. Schroeder, A. Chortos, A. Legrand, Z. Wang, J. Chang, P. Fox, Z. Bao, Nature Electronics, 1 (2018) 314-321.
[55] T. Numao, K. Ogawa, H. Fujinuma, N. Furuya, Journal of cardiology, 30 (1997) 1-8.
[56] J. T. Dodge Jr, B. G. Brown, E. L. Bolson, H. T. Dodge, Circulation, 86 (1992) 232-246.
[57] M. Back, G. Kopchok, M. Mueller, D. Cavaye, C. Donayre, R. A. White, Journal of vascular surgery, 19 (1994) 905-911.
[58] M. Elsisy, R. Herbert, W.-H. Yeo, J. J. Pacella, Y. Chun, Nano-, Bio-, Info-Tech Sensors and Wearable Systems, International Society for Optics and Photonics 2021, pp. 1159006.

1. A stent comprising:
a mechanical-sensing sensor comprising a plurality of flexible membrane members, including a first membrane member and a second membrane member, wherein the first membrane member is separated from the second membrane member across a dielectric member to form a capacitive structure, wherein the first membrane member is configured to move in a first direction in relation to the second membrane and the second membrane is configured to move in a second direction in relation to the first membrane member different from the first direction to change capacitance defined between the first membrane member and the second membrane member, and wherein the capacitance, or change of capacitance, corresponds to a measure of strain or mechanical properties; and
a stent body comprising a plurality of annular struts positioned at intervals in a circumferential direction of the stent body, wherein the mechanical-sensing sensor is coupled, via one or more stretchable interconnects, to the stent body to measure strain of the stent.

2. The stent of claim 1, wherein the plurality of annular struts include a first annular strut and a second annular strut, wherein the first annular strut is configured as an electromagnetic radiating body to serve as an antenna for the stent.

3. The stent of claim 2, wherein the second annular strut is configured as a second electromagnetic radiating body to serve as an antenna array for the stent with the first annular member.

4. The stent of claim 2, wherein the first annular strut is connected to a second annular strut via a plurality of non-conductive interconnects.

5. The stent of claim 2, wherein the first annular strut is configured as a wave-shaped strut.

6. The stent of claim 1, wherein the mechanical-sensing sensor is laminated on the stent body.

7. The stent of claim 1, wherein the mechanical-sensing sensor is integrated into the mechanical-sensing sensor.

8. The stent of claim 2, wherein each of the plurality of annular struts comprises a laminated structure comprising:
a metal core;
a conductive layer that surrounds the metal core; and
a coating.

9. The stent of claim 1, wherein the change of capacitance, strain, or mechanical property is employed to measure a state of restenosis of a patient.

10. The stent of claim 1, wherein the mechanical-sensing sensor is configured to measure strain or a change in strain.

11. The stent of claim 1, wherein the first membrane member has a first protruding structure, wherein the second membrane member has a second protruding structure, and wherein the first protruding structure is parallel to the second protruding structure.

12. The stent of claim 1, wherein the first membrane member has a plurality of conductive non-parallel members.

13. The stent of claim 1, wherein the stent was fabricated by
providing a substrate metal core;
cutting, via a laser operation, a plurality of bridges in the substrate metal core;
filling each of the plurality of bridges with a printed polyimide to form a stretchable interconnect;
cutting, the substrate metal core to form the plurality of annular struts positioned at intervals in a circumferential direction of the stent body;
electroplating the plurality of annular struts; and
coating the plurality of electroplated annular struts.

14. The stent of claim 1, wherein the mechanical-sensing sensor is fabricated by:
fabricating the first membrane member for a strain sensor;
fabricating the second membrane member for the strain sensor;
assembling the first membrane over a first side of a dielectric layer; and
assembling the second membrane over a second side of the dielectric layer.

15. The stent of claim 1, wherein the mechanical-sensing sensor is configured as
a strain sensor.

16.-17. (canceled)

18. A system comprising:
a measurement system comprising an antenna, an acquisition electronics, and a processing unit, wherein the processing unit comprises a processor a memory having instructions stored thereon, wherein execution of the instructions by the processor cause the processor to direct the acquisition electronics to measure and/or interrogate (i) a change in resonant frequency of an electronic stent implanted in a patient and/or (ii) a change in arterial wall strain properties, wherein the electronic stent includes
a mechanical-sensing sensor comprising a plurality of flexible membrane members, including a first membrane member and a second membrane member, wherein the first membrane member is separated from the second membrane member across a dielectric member to form a capacitive structure, wherein the first membrane member is configured to move in a first direction in relation to the second membrane and the second membrane is configured to move in a second direction in relation to the first membrane member different from the first direction to change capacitance defined between the first membrane member and the second membrane member, and wherein the capacitance, or change of capacitance, corresponds to a measure of strain or mechanical properties; and
a stent body comprising a plurality of annular struts positioned at intervals in a circumferential direction of the stent body, wherein the mechanical-sensing sensor is coupled, via one or more stretchable interconnects, to the stent body to measure strain of the stent.

19. (canceled)

20. A method of monitoring restenosis or progression of restenosis, the method comprising:
wirelessly interrogating an electronic stent implanted in a subject to determine a first resonant frequency associated with a strain or mechanical measurement, wherein the electronic stent comprises an inductive and a capacitive component;
wirelessly interrogating the electronic stent implanted in the subject to determine a second resonant frequency associated with the strain or mechanical measurement; and
determining a change the strain or mechanical measurement as a change between the first resonant frequency and the second resonant frequency to determine a presence of restenosis or the progression of restenosis.

21. The method of claim 20, wherein the wirelessly interrogation is continuously performed.

22. The method of claim 21, wherein the electronic stent includes a mechanical-sensing sensor comprising a plurality of flexible membrane members, including a first membrane member and a second membrane member, wherein the first membrane member is separated from the second membrane member across a dielectric member to form a capacitive structure, wherein the first membrane member is configured to move in a first direction in relation to the second membrane and the second membrane is configured to move in a second direction in relation to the first membrane member different from the first direction to change capacitance defined between the first membrane member and the second membrane member, and wherein the capacitance, or change of capacitance, corresponds to a measure of strain or mechanical properties; and
a stent body comprising a plurality of annular struts positioned at intervals in a circumferential direction of the stent body, wherein the mechanical-sensing sensor is coupled, via one or more stretchable interconnects, to the stent body to measure strain of the stent.
